(19) United States
(12) Patent Application Publication
(10) Pub. No.: US 2026/0002216 A1
(43) Pub. Date: Jan. 1, 2026

(54) Immune Classification Tool for Determining Survival Outcomes in Cancer

(71) Applicants: London Health Sciences Centre Research Inc., London (CA); The Regents of the University of California, Los Angeles, CA (US)

(72) Inventors: Yu Fan Zeng, Guelph (CA); Anthony Nichols, London (CA); John Barrett, London (CA); Paul Boutros, Marina del Ray, CA (US); Joseph Mymryk, London (CA)

(21) Appl. No.: 18/839,336

(22) PCT Filed: Feb. 18, 2023
(86) PCT No.: PCT/CA2023/050216

§ 371 Date: Aug. 16, 2024

Related U.S. Application Data
(60) Provisional application No. 63/311,732, filed on Feb. 18, 2022.

(51) Int. Cl. C12Q 1/6886

(52) U.S. Cl. CPC C12Q 1/6886; C12Q 2600/112; C12Q 2600/118; C12Q 2600/158

(57) ABSTRACT

An immune classification tool useful for determining survival outcomes in cancer patients based on the abundance in gene expression of CD3E, ZAP70 and IRF4 in a cancer tissue sample. The immune classification tool is able to stratify cancer patients, including HPV+HNSCC, HPV−HNSCC and cervical cancer patients, into the three immune groups (immune rich, immune desert, and mixed) that are strongly associated with disease-free survival.

### FIELD OF THE INVENTION

The present disclosure relates to an immune classification tool useful for determining survival outcomes in cancer patients. More particularly, to an immune classification tool based on the abundance of three gene transcripts that determines survival outcomes in cancer patients and that identifies cancer patients who respond to treatment de-escalation.

### BACKGROUND OF THE INVENTION

The incidence of human papillomavirus-driven (HPV⁺) head and neck squamous cell carcinoma (HNSCC) is increasing dramatically worldwide¹. HPV⁺ HNSCC is clinically distinct from non-HPV driven (HPV−) HNSCC, which is typically associated with tobacco and alcohol consumption²⁻⁴. However, both HPV⁺ and HPV⁻ HNSCC originate in same the anatomical regions. HPV⁺ HNSCC has further similarities with cervical cancer, which is also driven by HPV. Although HPV⁺ HNSCC patients are usually younger and exhibit markedly improved outcomes compared to HPV⁻ HNSCC patients³, current treatment guidelines from both American Society of Clinical Oncology (ASCO) and the National Comprehensive Cancer Network recommends identical treatment regimens of high-dose cisplatin and 70 Gy radiation (CRT) regardless of HPV status^{5,6}. The rising incidence of HPV⁺ OPSCC increasing number of young survivors, post-treatment quality of life is of particular importance as most patients will live with the substantial treatment-related morbidities for many years. Therefore, there is significant interest in treatment de-intensification for HPV⁺ HNSCC patients to reduce morbidity rates while maintaining the outstanding cure rates⁷⁻⁹. Treatment de-intensification efforts have been complicated by the ˜15-30% recurrence or metastasis rate of HPV⁺ HNSCC patients treated with the current standard of care therapy¹⁰. Early efforts at de-intensification demonstrated that modification of current standard of care CRT can result in harm for patients. Both the De-ESCALaTE HPV and RTOG1016 phase Ill randomized trials found that substitution of cisplatin for cetuximab led to inferior survival outcomes¹¹². Reflecting these unsuccessful attempts that led to poorer outcomes for patients on the experimental arm, recent treatment de-intensification guidelines from ASCO¹³ and the Head and Neck Cancer International Group¹⁴ have called for de-intensification to be only be attempted in the context of a clinical trial for patients with favorable risk profiles. Thus, the ideal treatment de-intensification method and patient population remain highly controversial^{5,13-18}.

Molecular biomarkers reflecting the biology of the tumour may better risk-identify patients who are ideal candidates for treatment de-intensification. HPV⁺ HNSCC treatment failure has been linked to TP53 mutations¹⁹, tumour hypoxia²⁰, keratinocyte differentiation²¹, chromosome 3p arm loss²², and HPV-related transcriptional programs²³. These findings have not yet been thoroughly validated, require complex assays only available at select institutions, or generally exhibit modest effect-sizes that explain only a fraction of HPV⁺ treatment failures. There remains an urgent need for improved risk-stratification to guide therapeutic decision-making in balancing treatment toxicity and therapeutic efficacy.

### SUMMARY OF THE INVENTION

In one embodiment, the present disclosure provides for a method for determining a prognosis for a cancer patient, the prognosis predicts a probability of disease-free survival for the cancer patient, the method comprising: (a) obtaining a sample having cancer cells from the cancer patient, (b) measuring gene expression levels of ZAP70, IRF4 and CD3E in the sample, (c) determining a tumor microenvironment (TME) subtype of the sample based on the gene expression levels of ZAP70, IRF4 and CD3E, the TME subtype being immune rich or non-immune rich, and (d) determining, based on the determined TME subtype, that the cancer patient has an increased likelihood of overall survival if the sample has an immune rich TME subtype.

In one embodiment of the method for determining a prognosis for a cancer patient, the non-immune rich TME subtype includes an immune mixed TME subtype and an immune depleted TME subtype, and wherein the sample having the immune depleted TME subtype indicates that the cancer patient has a poor likelihood of overall survival, and the sample having the immune mixed TME subtype indicates that the cancer patient has worse likelihood of survival than then immune rich TME subtype and better likelihood of survival than the immune depleted TME subtype.

In another embodiment of the method for determining a prognosis for a cancer patient, the method is done following a therapeutic treatment of the cancer in the cancer patient, and wherein the sample is obtained prior to or during the therapeutic treatment.

In another embodiment of the method for determining a prognosis for a cancer patient, the method further comprises determining an expression score of the sample based on the gene expression levels measured in (b), and wherein the TME subtype is determined based on the expression score of the test cancer sample.

In another embodiment of the method for determining a prognosis for a cancer patient, the gene expression levels of step (b) is obtained by counting a number cells within the sample that test positive for the presence of ZAP70, IRF4 and CD3E, and wherein said number of positive cells is used to generate the expression score of the sample.

In another embodiment of the method for determining a prognosis for a cancer patient, the gene expression levels of step (b) is obtained by measuring the levels of RNA transcript of ZAP70, IRF4 and CD3E.

In another embodiment of the method for determining a prognosis for a cancer patient, the expression score of the test cancer sample is determined using a known control reference value of each of the immune rich TME subtype and the non-immune rich TME subtype.

In another embodiment of the method for determining a prognosis for a cancer patient, the expression score of the sample is determined using formula I:

K1+abundance of ZAP70×K2+abundance of IRF4×K3+abundance of CD3E×K4 (I)

wherein K1 is a constant between 2 and 2.5, K2 is a constant between −0.2 and −0.5, K3 is a constant between −0.1 and −0.15 and K4 is a constant between −0.2 and −0.3, and
when the expression score of the sample is less or equal to 2 is indicative of the immune rich TME subtype, when the expression score of the sample is larger than 2 and less or equal to 2.5 is indicative of the immune mixed TME subtype, and when the expression score of the sample is larger than 2.5 is indicative of the immune depleted TME subtype.

In another embodiment of the method for determining a prognosis for a cancer patient, K1 is 2.23255813953488, K2 is −0.224179275535717, K3 is −0.137196384259042 and K4 is −0.273419927248369.

In another embodiment of the method for determining a prognosis for a cancer patient, step (b) further comprises measuring gene expression level of WDFY4 in the sample, and in step (c) determining the TME subtype of the sample is based on the gene expression levels of ZAP70, IRF4, CD3E and WDFY4.

In another embodiment of the method for determining a prognosis for a cancer patient, when the sample is determined to be immune depleted TME subtype, the method further comprises treating the cancer patient with at least one of chemotherapy, radiotherapy, and immunotherapy suitable for a poor prognosis.

In another embodiment of the method for determining a prognosis for a cancer patient, when the sample is determined to be immune rich TME subtype, the method further comprises treating the cancer patient with at least one of chemotherapy, radiotherapy and immunotherapy suitable for the favorable prognosis.

In another embodiment of the method for determining a prognosis for a cancer patient, the cancer is human papillomavirus-driven (HPV⁺) head and neck squamous cell carcinoma (HNSCC), non-human papillomavirus driven (HPV−) HNSCC, endometrial cancer or cervical cancer.

In another embodiment of the method for determining a prognosis for a cancer patient, the cancer is human papillomavirus-driven (HPV⁺) head and neck squamous cell carcinoma (HNSCC).

In another embodiment, the present disclosure relates to a method for determining a prognosis for a cancer patient, the method comprising: (a) obtaining a sample comprising cancer cells from the cancer patient, (b) measuring gene expression levels of ZAP70, IRF4 and CD3E in the sample, and (c) determining a tumor microenvironment (TME) subtype of the sample by comparing the gene expression levels of (b) with known abundance reference gene expression values of ZAP70, IRF4 and CD3E in an immune rich TME subtype, and in a non-immune rich TME subtype, wherein the prognosis predicts a probability of disease-free survival for the cancer patient, and wherein determining that the test cancer tissue sample has the immune rich TME subtype indicates a favorable prognosis, and determining that the test cancer tissue sample has the non-immune rich TME subtype indicates a poor prognosis.

In one embodiment of the method for determining a prognosis for a cancer patient, the non-immune rich TME subtype includes an immune mixed TME subtype and an immune depleted TME subtype, and wherein the immune depleted TME subtype indicates a poor prognosis and the immune mixed TME subtype indicates worse prognosis than the immune rich TME subtype and better prognosis than the immune depleted TME subtype.

In another embodiment of the method for determining a prognosis for a cancer patient, step (b) further comprises measuring gene expression levels of WDFY4 in the sample, and step (c) comprises comparing the gene expression levels of (b) with known abundance reference gene expression values of ZAP70, IRF4, CD3E and WDFY4 in control samples of the immune rich TME subtype, the immune mixed TME subtype and the immune depleted TME subtype.

In another embodiment of the method for determining a prognosis for a cancer patient, the gene expression levels are obtained by counting the number of cells in the sample that stain positive for the presence of CD3, ZAP70 and IRF4.

In another embodiment of the method for determining a prognosis for a cancer patient, the gene expression levels are obtained by counting the number of cells in the sample that stain positive for the presence of CD3, ZAP70, IRF4 and WDFY4.

In another embodiment of the method for determining a prognosis for a cancer patient, the gene expression levels are obtained by measuring the levels of RNA transcript of ZAP70, IRF4 and CD3E.

In another embodiment of the method for determining a prognosis for a cancer patient, the gene expression levels are obtained by measuring the levels of RNA transcript of ZAP70, IRF4, CD3E and WDFY4.

In another embodiment of the method for determining a prognosis for a cancer patient, when the sample is determined to be immune depleted TME subtype, the method further comprises treating the cancer patient with at least one of chemotherapy, radiotherapy, and immunotherapy suitable for a poor prognosis.

In another embodiment of the method for determining a prognosis for a cancer patient, when the sample is determined to be immune rich TME subtype, the method further comprises treating the cancer patient with at least one of chemotherapy, radiotherapy and immunotherapy suitable for the favorable prognosis.

In another embodiment of the method for determining a prognosis for a cancer patient, the cancer is human papillomavirus-driven (HPV⁺) head and neck squamous cell carcinoma (HNSCC), non-human papillomavirus driven (HPV⁻) HNSCC, endometrial cancer or cervical cancer.

In another embodiment of the method for determining a prognosis for a cancer patient, the cancer is human papillomavirus-driven (HPV⁺) head and neck squamous cell carcinoma (HNSCC).

In another embodiment, the present disclosure provides a method of treating a cancer patient, the method comprising: (a) determining a tumor microenvironment (TME) subtype of the cancer patient based on gene expression levels of ZAP70, IRF4 and CD3E in a cancer tissue sample of the patient, the TME subtype being immune rich and non-immune rich, and (b) administering to the cancer patient a cancer treatment suitable for a favourable prognosis when the cancer patient has the immune rich TME subtype or administering to the cancer patient a cancer treatment suitable for a poor prognosis when the cancer patient has the non-immune rich TME subtype.

In one embodiment of the method of treating a cancer patient, the TME subtype is determined based in the gene expression levels of ZAP70, IRF4, CD3E and WDFY4.

In another embodiment of the method of treating a cancer patient, the cancer treatment includes at least one of chemotherapy, radiotherapy and immunotherapy.

In another embodiment of the method of treating a cancer patient, the cancer is human papillomavirus-driven (HPV⁺) head and neck squamous cell carcinoma (HNSCC), non-human papillomavirus driven (HPV⁻) HNSCC, endometrial cancer or cervical cancer.

In another embodiment, the present disclosure relates to a computer program product for use in conjunction with a computer system having one or more processors, the computer program product comprising a non-transitory computer readable storage medium and one or more computer programs embedded therein for execution by the one or more processors, the one or more computer programs comprising instructions for performing a method for determining a prognosis for a cancer patient in a subject, the prognosis predicts a probability of disease-free survival for the cancer patient, the method comprising: (a) obtaining values in gene expression levels of ZAP70, IRF4 and CD3E in a sample having cancer cells obtained from the cancer patient, (b) determining, from the values obtained in (a), a plurality of data elements for the test cancer tissue sample, and (c) applying, to the plurality of data elements a model that is trained to provide a tumor microenvironment (TME) subtype for the sample, the TME subtype being immune rich, immune mixed or immune depleted, wherein when model provides the immune rich TME subtype indicates a favorable prognosis for the cancer patient, when the model provides the immune depleted TME subtype indicates a poor prognosis for the cancer patient and when the model provides the immune mixed TME subtype indicates worse prognosis than the immune rich TME subtype and better prognosis than the immune depleted TME subtype for the cancer patient.

In one embodiment of the computer system of the present disclosure, the values of step (a) further comprise the gene expression levels of WDFY4.

In another embodiment of the computer system of the present disclosure, the cancer is human papillomavirus-driven (HPV⁺) head and neck squamous cell carcinoma (HNSCC), non-human papillomavirus driven (HPV⁻) HNSCC, endometrial cancer or cervical cancer.

In another embodiment, the present disclosure relates to a method for planning a treatment for a cancer patient, the method comprising: (a) providing a cancer patient having an immune rich tumor microenvironment (TME) subtype or providing a patient having a non-immune rich TME subtype, the TME being based on gene expression levels of ZAP70, IRF4 and CD3E in a cancer tissue sample of the patient, and (b) referring the cancer patient to a cancer treatment suitable for a favourable prognosis when the cancer patient has the immune rich TME subtype or administering to the cancer patient a cancer treatment suitable for a poor prognosis when the cancer patient has the non-immune rich TME subtype.

In one embodiment of the method for planning a treatment for a cancer patient, the TME subtype is determined based in the gene expression levels of ZAP70, IRF4, CD3E and WDFY4.

In another embodiment of the method for planning a treatment for a cancer patient, the cancer treatment includes at least one of chemotherapy, radiotherapy and immunotherapy.

In another embodiment of the method for planning a treatment for a cancer patient, the non-immune rich subtype includes an immune mixed TME subtype and an immune depleted TME subtype.

In another embodiment of the method for planning a treatment for a cancer patient of the present disclosure, the cancer is human papillomavirus-driven (HPV⁺) head and neck squamous cell carcinoma (HNSCC), non-human papillomavirus driven (HPV⁻) HNSCC, endometrial cancer or cervical cancer.

### BRIEF DESCRIPTION OF THE DRAWINGS

The following figures illustrate various aspects and preferred and alternative embodiments of the disclosure.

FIGS. 1A-1F. UWO3 immune group is a strong predictor of survival outcomes across six independent cohorts. Patients from the immune desert and mixed group show inferior disease-free survival and overall survival compared to the immune rich patients (1A-1D). Hazard ratio (HR) are based on the univariate Cox model and were combined using Mantel-Haenszel fixed-effect model. Heterogeneity between studies was analyzed with χ² and I² statistics. * Hazard ratio for overall survival in the JHU cohort excluded from the analysis due to only one event in the cohort. Pooled Kaplan-Meier analyses of disease-free survival (1E) and overall survival (1F) of HPV⁺ HNSCC patients show that UWO3 immune groups are associated with distinct survival outcomes. P values from two-sided log rank tests and Cox proportional regression model.

FIGS. 2A-2B. UWO3 immune class outperforms clinical factors in predicting disease-free survival. Brier prediction score analysis (2A) shows lower error rate, thus higher prediction accuracy of disease-free survival for the UWO3 immune class than major clinical factors combined (AJCC8 stage, age, sex, smoking status). Integration of UWO3 immune group with other clinical factors further decreased prediction error rate. Relative importance of each risk (2B) parameter to survival risk using the Pearson X test for clinical parameters plus UWO immune group shows that immune group is the most important factor. AJCC: American Joint Committee on Cancer.

FIGS. 3A-3B. UWO3 immune classification has implications for immunotherapy and treatment de-intensification. 3A. Tumours from UWO3 immune desert group exhibit increased relative abundance of T-cell co-inhibitory receptors HAVCR2 (encodes TIM-3) and LAG3. The abundance of co-inhibitory receptors for each sample was normalized to T-cell abundance in the tumour. FDR values from Benjamini-Hochberg corrected two-sided Wilcoxon signed-rank test. *: FDR<0.05, **: FDR<0.01, ***: FDR<0.001. ****: FDR<0.0001. 3B. UWO3 score can identify patients to aggressive radiation de-escalation from 70 grays (Gy) to 30 Gy in the Mayo Clinic MC1273 trial (NCT01932697) and the Memorial Sloan Kettering (MSK, NCT00606294) 30ROC trial. Recurrence is defined as patients who have not developed locoregional or metastatic disease as of last follow-up. Odds ratio and P-value are from logistic regression with UWO3 as a continuous variable and stratified for cohort.

FIG. 4. UWO3 immune classification of HPV⁺ HNSCC with implications for treatment de-intensification and immunotherapy. CI: confidence interval; DFS: disease-free survival; OS: overall survival.

FIGS. 5A-5C. Immune classification of HPV⁺ HNSCC. Composition of the HPV⁺ HNSCC TME (5A) as defined by MCP-counter generated Z-score, immune gene signature scores, and expression immune checkpoint related genes. Clustering based on K-means clustering of MCP-counter estimated immune abundance Z-score. Q values derived from Benjamini-Hochberg corrected Kruskal-Wallis P values. Kaplan-Meier analyses of overall survival (5B) and disease-free survival (5C) of HPV⁺ oropharyngeal cancer patients by their tumor immune group show distinct survival patterns. All tests are two-sided log-rank tests.

FIG. 6. Derivation and validation of the UWO3 score.

FIGS. 7A-7C. UWO3 immune groups predict treatment response in two external cohorts. Kaplan-Meier analyses of disease-free survival of HPV⁺ HNSCC patients in the LHSC (7A), TCGA (7B), and JHU (7C) cohort by their immune group as assigned through the UWO3 score demonstrate that UWO3 immune groups can predict survival. P values from two-sided log rank tests and Cox proportional regression model.

FIG. 8A-8B. UWO3 immune groups predict recurrence in a cohort of HPV⁺ HNSCC patients using immunohistochemistry. Representative TMA images stained for hematoxylin and eosin (H&E), p16 (a surrogate marker of HPV status), and the three proteins used to calculate UWO3 (CD3E, ZAP70, and IRF4, A). Kaplan-Meier analyses of the disease-free survival (8B) of the tumour microarray (TMA) demonstrate that UWO3 immune groups defined using clinically-validated antibodies can predict recurrence in HPV⁺ HNSCC. P value from Cox proportional regression model.

FIGS. 9A-9D. Blinded external validation of the UWO3 immune groups in a retrospective cohort and a prospective cohort. Kaplan-Meier analyses of disease-free survival (9A) and overall survival (9B) of HPV⁺ HNSCC patients in a retrospective cohort of patients from the Washington University at St. Louis (WashU) and Vanderbilt University shows that UWO3 immune groups are associated with distinct survival outcomes. Further, in a prospective cohort (BD2Decide, NCT02832102) where HPV⁺ HNSCC patients received uniform treatment, Kaplan-Meier analyses of disease-free survival (9C) and overall survival (9D) shows that UWO3 immune groups are associated with distinct survival outcomes. P values from two-sided log rank tests and Cox proportional regression model.

FIGS. 10A-10D. UWO3 score can predict survival outcomes independent of primary treatment. Kaplan-Meier analyses of disease-free survival (10A) and overall survival (10B) of HPV⁺ HNSCC patients treated with primary surgical approach with or without adjuvant therapy. Kaplan-Meier analyses of disease-free survival (10C) and overall survival (10D) of HPV⁺ HNSCC patients treated with primary radiation with or without chemotherapy. P values from univariate Cox proportional regression model.

FIG. 11. UWO3 score can predict survival outcomes in cancer types other than HPV⁺ HNSCC. We have identified CD3E, IRF4, and ZAP70 to be prognostic in other cancer types including HPV-negative HNSCC, cervical cancer, and endometrial cancer. The biology of these tumour types are similar to HPV-positive HNSCC, thus the UWO3 may also allow improved risk-stratification in these tumour types.

### DETAILED DESCRIPTION

### Abbreviations

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The contents of all documents (including patent documents and non-patent literature) cited in this application are incorporated herein by reference.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

The term “subject” as used herein refers all members of the animal kingdom including mammals, preferably humans.

The term “patient” as used herein refers to a subject that has been diagnosed as having cancer.

A patient may be considered to have a “good prognosis” where, for example, the survival rate associated with the cancer subtype is greater than a survival rate associated with other related cancer subtypes. In certain embodiments, a “good prognosis” indicates at least an increased expected survival time. This may be based upon a classification as responsive to an anti-angiogenic therapeutic agent as described herein. The increased expected survival time may be as compared to classification as non-responsive to the anti-angiogenic therapeutic agent.

A patient may be considered to have a “poor prognosis” or “bad prognosis” where, for example, the survival rate associated with the cancer subtype is less than the survival rate associated with other related cancer subtypes.

A cancer is “responsive” to a therapeutic agent if its rate of growth is inhibited as a result of contact with the therapeutic agent, compared to its growth in the absence of contact with the therapeutic agent. Growth of a cancer can be measured in a variety of ways. For instance, the size of a tumor or measuring the expression of tumor markers appropriate for that tumor type.

A cancer is “non-responsive” to a therapeutic agent if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or measuring the expression of tumor markers appropriate for that tumor type. The quality of being non-responsive to a therapeutic agent is a highly variable one, with different cancers exhibiting different levels of “non-responsiveness” to a given therapeutic agent, under different conditions. Still further, measures of non-responsiveness can be assessed using additional criteria beyond growth size of a tumor such as, but not limited to, patient quality of life, and degree of metastases.

“Gene expression levels” refers to the amount of mRNA transcribed by a gene or to the amount of protein encoded by a gene in a sample. Detecting gene expression levels can be achieved using any method known in the art or described herein, such as by qRT-PCR or by histochemistry. In this document, the term “abundance” is used to refer to “gene expression levels.”

In this document, the term “oropharyngeal cancer” and “head and neck cancer” are used interchangeable. As such, the term “human papillomavirus-driven (HPV⁺) head and neck squamous cell carcinoma (HNSCC)” can be used interchangeably with “HPV⁺ oropharyngeal squamous cell carcinoma (OPSCC)”.

### Overview

Presented herein is a clinically translatable immune classification tool associated with survival outcomes in cancer patients, including HPV⁺ HNSCC, HPV⁻ HNSCC and cervical cancer, based upon the abundance of three gene transcripts. Analysis of tumour tissue samples revealed three distinct tumor microenvironment (TME) subtypes: immune rich, immune desert, and mixed. In one embodiment, a three gene immune score (referred to in this disclosure as “UWO3”), based on the abundance in gene expression (i.e., gene expression levels) of CD3E, ZAP70 and IRF4 in a cancer tissue sample, was able to stratify patients into the three immune groups and was strongly associated with disease-free survival.

Pooled analysis of all cohorts (n=906) demonstrated that patients in the immune rich subtype had superior disease-free survival at 5 years (87.2% immune rich vs. 73.4% mixed vs. 63.8% immune desert. The association between UWO3 immune class and time to recurrence was independent of patient age, sex, smoking status, alcohol intake status, and AJCC 8th edition clinical stage (P<0.05). Moreover, the immune classification tool presented herein serves to identify patients who respond to aggressive treatment de-escalation. Lastly, immune desert or depleted patients have distinct expression patterns of immune checkpoint receptors.

The immune classification tool can be used with any suitable technique that measures the abundance of CD3E, IRF4, and ZAP70 gene transcripts or CD3E, IRF4, and ZAP70 proteins in cancer cells. In one embodiment, the immune classification tool can be used by measuring, for example by microarray assay, the expression levels of CD3E mRNA, ZAP70 mRNA and IRF4 mRNA. In embodiments, the immune classification can be assessed using immunohistochemistry. Antibodies against CD3E, ZAP70, and IRF4 are routinely used in clinical pathology labs and assessment by immunohistochemistry in a cohort of cancer patients using tumour microarray was also predictive of disease-free survival.

In another embodiment, the immune classification tool further comprises measuring the gene expression levels of WDFY4.

In one embodiment, the present disclosure provides for a method for determining a prognosis for a cancer patient, the method comprising: (a) obtaining a test cancer tissue sample from the patient, (b) measuring the abundance in gene expression (i.e., the gene expression levels) of ZAP70, IRF4 and CD3E in the test cancer tissue sample, and (c) determining a tumor microenvironment (TME) subtype of the test cancer tissue sample based on the abundance in the gene expression of ZAP70, IRF4 and CD3E, the TME subtype being immune rich and non-immune rich. The non-immune rich TME subtype further including immune mixed TME subtype or immune depleted TME subtype. The prognosis predicts a probability of disease-free survival for the patient. Determining that the test cancer tissue sample has an immune rich TME subtype indicates a favorable prognosis for the patient. A determination that the test cancer tissue sample has an immune depleted TME subtype indicates a poor prognosis for the patient. A determination that the test cancer tissue sample has an immune mixed TME subtype indicates worse prognosis than the immune rich TME subtype and better prognosis than the immune depleted TME subtype.

In one embodiment, a library of abundance values of CD3E, ZAP70 and IRF4 gene expression for each TME subtype (i.e., immune rich, immune mixed and immune depleted) is created. This library is used to obtain reference threshold values for each TME subtype. A determination of the TME subtype can be obtained by comparing the abundance in the gene expression of CD3E, ZAP70 and IRF4 in a test cancer tissue sample obtained from a patient to the known threshold abundance values of each TME subtypes.

In another embodiment, RNA-abundance for each of the CD3E, ZAP70 and IRF4 genes in a test cancer sample is used to calculate a test cancer sample score (UWO3) using formula I:

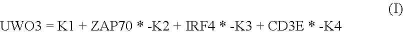

wherein, K1 ranges between 2 and 2.5, K2 ranges between 0.2 and 0.5, K3 ranges between 0.1 and 0.15 and K4 ranges between 0.2 and 0.3.

In one embodiment, K1 is 2.23255813953488, K2 is 0.224179275535717, K3 is 0.137196384259042 and K4 is 0.273419927248369.

Using UWO3, tumours are then assigned to an TME subtype as follow: UWO3<=2 “immune rich”, 2<UWO3<=2.5 “immune mixed”, and UWO3>2.5 “immune desert”.

The prognosis predicts a probability of disease-free survival for the patient. Determining that the test cancer tissue sample obtained from a cancer patient has an immune rich TME subtype indicates a favorable prognosis for the patient, in which case the immune rich patient can be treated with radiotherapy and/or chemotherapy suitable for a favourable diagnosis. This can include de-escalating or decreasing the immunotherapy, radiotherapy and/or chemotherapy that the patient was receiving.

In another embodiment, the present disclosure relates to a method of planning a treatment of a cancer patient based on the TME subtype. Determining that the test cancer tissue sample has an immune depleted TME subtype indicates a poor prognosis for the patient. In this case the immune depleted patient can be treated with radiotherapy and/or chemotherapy suitable for a poor diagnosis. This can include escalating or increasing the immunotherapy, radiotherapy and/or chemotherapy that the patient was receiving.

Determining that the test cancer tissue sample has an immune mixed TME subtype indicates worse prognosis than the immune rich TME subtype albeit better prognosis than the immune depleted TME subtype. In this case the immune mixed patient can be treated with immunotherapy, radiotherapy and/or chemotherapy suitable for a poor diagnosis. This can include escalating or increasing the radiotherapy and/or chemotherapy that the patient was receiving.

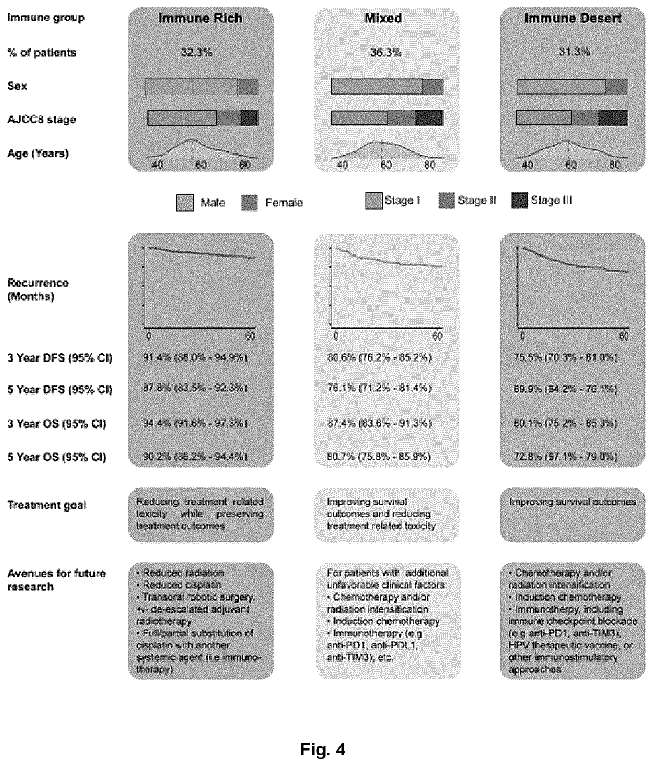

Treatment for each TME subtype identified by the scoring system of the present disclosure is described in FIG. 4.

Measurements of the abundance in gene expression of CD3E, ZAP70 and IRF4 in the methods described herein can be determined by measuring the levels of RNA transcripts of CD3E, ZAP70 and IRF4, or by measuring the levels of expressed nucleic acids encoding proteins (i.e., the levels of CD3E, ZAP70 and IRF4 proteins) or by using immunohistochemistry techniques using antibodies against CD3E, ZAP70 and IRF4 and counting cells in a tumor tissue sample that are positive for CD3E, ZAP70 and IRF4 staining. The percentage of positive cells within each tumour is used to create Z-score and used to generate the UWO3 score. Using UWO3, tumours are then assigned to an immune class as follow: UWO3<=2 were predicted be “immune rich”, 2<UWO3<=2.5 were predicted to be “mixed”, and UWO3>2.5 were predicted to be “immune desert”.

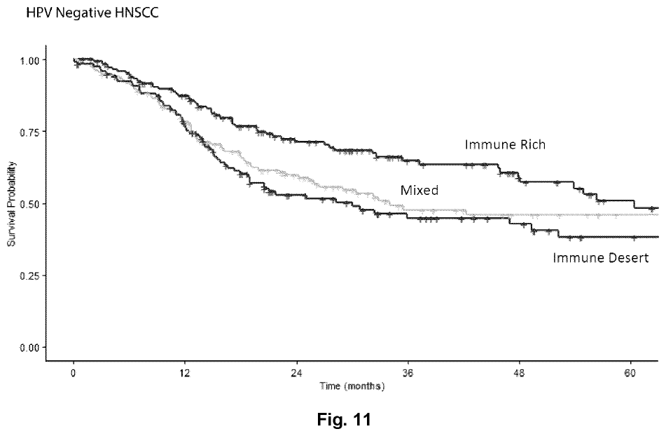

The embodiments of the present invention can be used to obtain the prognosis of any cancer. As shown in FIG. 11, the tools of the present disclosure can be used to obtain the prognosis of at least human papillomavirus-driven (HPVP⁺P) head and neck squamous cell carcinoma (HNSCC), non-human papillomavirus driven (HPV⁻) HNSCC, endometrial cancer or cervical cancer.

In another embodiment the present disclosure provides for a computer program product for use in conjunction with a computer system having one or more processors, the computer program product comprising a non-transitory computer readable storage medium and one or more computer programs embedded therein for execution by the one or more processors, the one or more computer programs comprising instructions for performing a method for determining a prognosis for a cancer patient in a subject, the prognosis predicts a probability of disease-free survival for the cancer patient, the method comprising: (a) obtaining values in gene expression levels of ZAP70, IRF4 and CD3E in a test cancer tissue sample obtained from the cancer patient, (b) determining, from the values obtained in (a), a plurality of data elements for the test cancer tissue sample, and (c) applying, to the plurality of data elements a model that is trained to provide a tumor microenvironment (TME) subtype for the test cancer tissue sample, the TME subtype being immune rich, immune mixed or immune depleted, wherein when model provides the immune rich TME subtype indicates a favorable prognosis for the cancer patient, when the model provides the immune depleted TME subtype indicates a poor prognosis for the cancer patient and when the model provides the immune mixed TME subtype indicates worse prognosis than the immune rich TME subtype and better prognosis than the immune depleted TME subtype for the cancer patient. In one embodiment, the values in gene expression levels obtained in step (a) further comprise the gene expression levels of WDFY4.

### EXAMPLES

The examples are described for the purposes of illustration and are not intended to limit the scope of the disclosure.

### Example 1

Presented herein is a clinically translatable immune classification tool that strongly associated with survival outcomes in HPV⁺ HNSCC based upon the abundance of three transcripts. The tool of the present disclosure has been validated it in five HPV+ HNSCC cohorts comprising 863 patients, including two blinded cohorts and a tissue microarray (TMA) cohort using immunohistochemistry. The immune classification of the present disclosure can identify patients who responded to aggressive treatment de-escalation. Taken together, in one embodiment, the present disclosure enables biomarker-guided personalized treatment deintensification and intensification in HPV+ HNSCC low and high-risk groups respectively.

### Ethics

The study was approved by the Research Ethics Boards at Western University (REB 7182) and informed consent was obtained from each patient. Primary site fresh tumour samples were prospectively collected from patients with HPV⁺ oropharyngeal squamous cell cancer at Victoria Hospital, London Health Science Centre, London, Ontario, Canada between 2010 and 2016.

### Patient Cohort

Patient demographics and survival outcomes were prospectively collected. Frozen section analysis was carried out to confirm tumor cellularity greater than 70%. HPV status was confirmed via p16 immunohistochemistry as well as PCR and Sanger sequencing. Detailed clinical information in the LHSC cohort is provided in Table 1. Disease free survival was defined as time from diagnosis to recurrence at any site or death. Recurrence was defined as the presence of local, regional, or distant disease after completion of treatment. The Cancer Genome Atlas (TCGA, n=71), Johns Hopkins University (JHU; n=47), BD2Decide (n=286), and Washington University (WashU) and Vanderbilt University (n=262) cohorts are public HPV⁺ HNSCC detailed elsewhere^{30-32,52,53}. Treatments received for each cohort are described in Table 2. The reporting of the study followed guidelines from Reporting Recommendations for Tumor Marker Prognostic Studies (REMARK)⁵⁴.

### RNA-Seq Processing, Sequencing, and Bioinformatics

### RNA Processing and Sequencing

Total RNA and DNA was isolated using Qiagen AllPrep DNA/RNA kits. HPV status was confirmed by real time PCR as we have previously described^{1,2}. One microgram of total RNA was shipped to The Center for Applied Genomics (Hospital for Sick Children, Toronto, ON) for quality control, library preparation, and sequencing. RNA quality was confirmed with a Bioanalyzer and libraries were prepared using a NEB Ultra II Directional mRNA library kit. Samples were then processed using random primers and sequenced using an Illumina HiSeq 2500 paired end for 50-90 million reads/sample (median: 66 million reads).

FASTQ files were pre-processed with trim_galore (v0.6.4) and then quality controlled using FastQC (v0.11.9). Each sample were mapped to the human reference genome GRCh38 (v97) using STAR aligner (v2.7.2b) in two-pass mode³, and quantified using HTSeq-count (v0.12.3) intersection-strict mode⁴. Read normalization and differentially expressed gene testing was conducted using DESeq2 (v1.26.0)⁵. Differentially abundant transcripts between the disease-free and recurrent patient are defined as transcripts having at least an average of normalized reads of 10, an absolute log 2 fold change (log₂FC) greater than 2, and a Benjamini-Hochberg adjusted p-value of less than 0.05. Generation and processing of external cohorts have been described elsewhere⁶⁻¹⁰.

### HPV Genotyping Via HPV Transcript Quantification

HPV genotyping were performed on raw RNA-seq reads using HPViewer (branch c62f29e, available at https://github.com/yuhanH/HPViewer), on a database of 182 repeat masked HPV strains. HPV reads were then quantified using HTSeq-count intersection-strict mode to the subtype with the highest read-number¹¹.

### TME Estimation

The tumour microenvironment (TME) composition of each sample was estimated using the MCP-counter score (v1.1.0)¹². The score was based on previously analyzed transcriptomic markers that are found to be characteristic of the specific immune population and were proportional to the abundance of each cell population within the tumour. Comparison with other immune deconvolution methods have found the method to be highly accurate and capable of inter-sample comparisons¹³. The MCP-counter signatures composition are as follows: T cells: CD28, CD3D, CD3G, CD5, CD6, CHRM3-AS2, CTLA4, FLT3LG, ICOS, MAL, PBX4, SIRPG, THEMIS, TNFRSF25 and TRAT1; B lineage: BANK1, CD19, CD22, CD79A, CR2, FCRL2, IGKC, MS4A1 and PAX5; natural killer cells: CD160, KIR2DL1, KIR2DL3, KIR2DL4, KIR3DL1, KIR3DS1, NCR1, PTGDR and SH2D1B; monocytic lineage: ADAP2, CSF1R, FPR3, KYNU, PLA2G7, RASSF4 and TFEC; myeloid dendritic cells: CD1A, CD1B, CD1E, CLEC10A, CLIC2 and WFDC21P; neutrophils: CA4, CEACAM3, CXCR1, CXCR2, CYP4F3, FCGR3B, HAL, KCNJ15, MEGF9, SLC25A37, STEAP4, TECPR2, TLE3, TNFRSF10C and VNN3; endothelial cells: ACVRL1, APLN, BCL6B, BMP6, BMX, CDH5, CLEC14A, CXorf36 (also known as DIPK2B), EDN1, ELTD1, EMCN, ESAM, ESM1, FAM124B, HECW2, HHIP, KDR, MMRN1, MMRN2, MYCT1, PALMD, PEAR1, PGF, PLXNA2, PTPRB, ROBO4, SDPR, SHANK3, SHE, TEK, TIE1, VEPH1 and VWF.

### Clustering

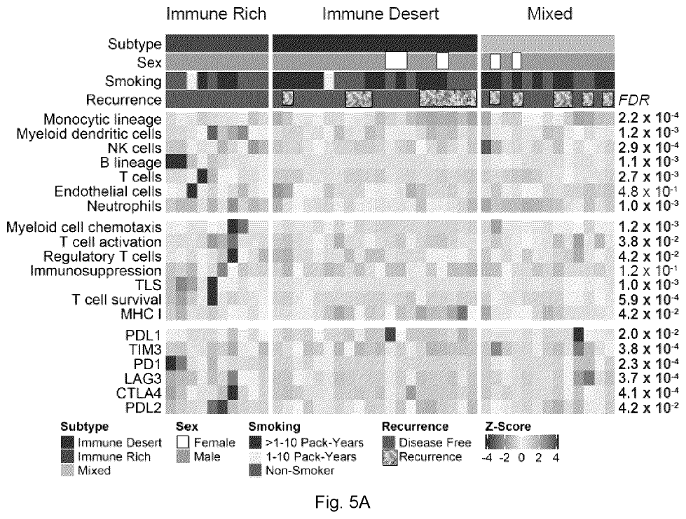

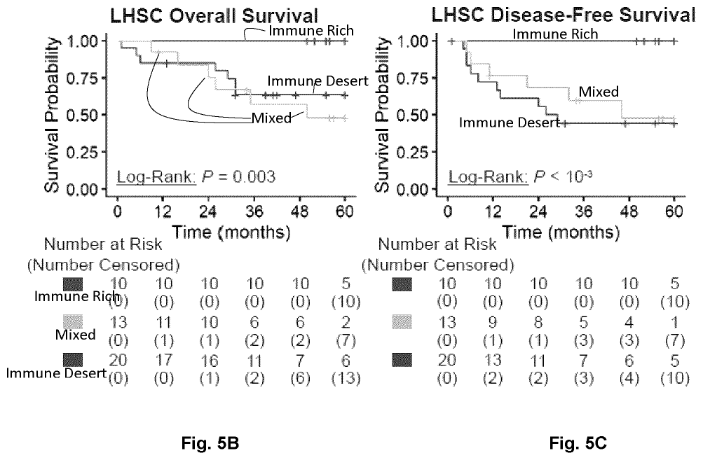

K-means clustering was performed on Z-scores of the immune cell abundance estimation (FIG. 5A) using the kmeans wrapper of Complex-Heatmap package (v2.1.0) when generating heatmap¹⁴. Kmeans was run 1000 times to generate consensus k-means clustering. The number of K clusters was selected using the silhouette method through the fviz_nbclust function of the factoextra R package (v1.0.6).

### Immune Gene Signatures

Immune gene signatures were derived from other studies¹⁵. Briefly, each signatures were computed as geometric mean of the abundance of its included genes: immunosuppression (TGFB1, TGFB3, LGALS1, and CXCL12), regulatory T cells (FOXP3, TNFRSF18), T cell survival score (CD70 and CD27), T cell activation (CXCL9, CXCL10, CXCL16, IL15, and IFNG), myeloid chemotaxis (CCL2), MHC I (HLA-A, HLA-B, HLA-C, HLA-E, HLA-F, HLA-G, and B2M), and tertiary lymphoid structures (CXCL13).

### UWO3 Score Development and Prediction

Statistically significant transcripts between disease-free and recurrent tumour were filtered to only include transcripts whose abundance is independently associated with prognosis. The cohort was dichotomized into high and low abundance groups for each transcript and tested for association with prognosis in a Cox proportional hazard multivariate model including important clinical variables (age at diagnosis, sex, T stage, N stage, smoking status, alcohol abuse status). Transcripts with false discovery rate (FDR)<0.25 had it abundance added 0.1, log-10 transformed, and scaled for score development. To facilitate the development of protein diagnostic tools for pathology, the gene lists were further filtered to contain genes for which the RNA abundance is highly correlated with its protein abundance (FDR<0.05 & rho>0.6, Spearman Correlation) using the HPV-negative HNSCC CPTAC cohort¹⁶.

For the development of UWO3 score to differentiate between the 3 TME signatures, we used a regularized linear regression technique based on the LASSO algorithm as implemented in the glmnet (v3.0-2) R package using 5-fold cross-validation. The score was trained with “gaussian” family of model to minimize mean squared error (MSE). A minimal subset of 3 genes (CD3E, IRF4, and ZAP70) was selected whose weighted RNA abundance (UWO3 score) was highly associated with survival outcomes in the training cohort. RNA-abundance for each of the gene is then log₁₀, scaled within the cohort, and used to calculate the UWO3 score with the following equation: UWO3=2.23255813953488+ZAP70*−0.224179275535717+IRF4*−0.137196384259042+CD3E*−0.273419927248369. Using UWO3, tumours are then assigned to an immune class as follow: UWO3<=2 were predicted be “immune rich”, 2<UWO3<=2.5 were predicted to be “mixed”, and UWO3>2.5 were predicted to be “immune desert”. Cut offs were determined by maximizing the correct number of tumours assigned to the same immune subtype through both clustering and the UWO3 and ensuring similar number of patients between each group.

### Tumour Microarray (TMA) and Immunohistochemistry

All samples were obtained with informed consent after approval of the Institutional Review Board at Western University, the University of Calgary, and the University of British Columbia. The TMA from University of Calgary and University of British Columbia have been described previously^{9,17}. The TMA from Western University are processed as following. The formalin-fixed paraffin-embedded (FFPE) blocks for each tumor was sectioned and stained with hematoxylin & eosin (H&E) to confirm the presence of human tumor. A Manual Tissue Arrayer (MTA-1; Beecher Instruments Inc.) was used to punch out 3-4 cylindrical cores of 0.6 mm diameter from each tumour sample. Cores were arrayed into recipient paraffin blocks. Control tissues were also included on each block. Cores were sealed into recipient blocks by heating at 40° C. for ˜40 mins. Blocks were sectioned into 1.5 μM sections and affixed to glass slides. Every ninth slide was stained with H&E to provide a reference. Additional details are available in the MTA-1 Instruction Manual (www.beecherinstruments.com). IHC staining was completed at the Department of Pathology & Laboratory Medicine and the Molecular Pathology Core Facility (Western University). Tissues were examined using an Aperio ScanScope® slide scanner and staining quantification was performed using the QuPath (v0.2.3).

To translate the UWO3 score for use in TMAs, we stained the TMA with anti-CD3 (IR503, Agilent Dako), anti-ZAP70 (clone 2F3.2, IR653, Agilent Dako), and anti-IRF4 (clone MUM1p, IR644, Agilent Dako) antibodies on an Omnis staining platform (Agilent Dako). The tumor was contoured by a subspecialist pathologist and the number of cells within the tumour positive for each marker was quantified using the positive cell detection function in QuPath¹⁸. The percentage of positive cells within each tumour was then used to create Z-score and used to generate the UWO3 score. Using UWO3, tumours are then assigned to an immune class as follow: UWO3<=2 were predicted be “immune rich”, 2<UWO3<=2.5 were predicted to be “mixed”, and UWO3>2.5 were predicted to be “immune desert”.

### T-Cell Co-Receptor Analysis

To analyze the relative abundance of co-receptors on T-cells, we normalized the abundance of the co-receptors (CTLA4, PDCD1, ICOS, TIGIT, LAG3, and HAVCR2) to the abundance of T-cells. In the RNA-seq cohorts, the relative abundance was calculated by dividing the abundance of each co-receptor by the abundance of CD3E. In the BD2Decide microarray cohort, the relative abundance was calculated by subtracting the intensity of CD3E from the intensity of each co-receptor.

### REFERENCES FOR RNA-SEQ PROCESSING, SEQUENCING, AND BIOINFORMATICS

1. Mundi N, Prokopec S D, Ghasemi F, et al: Genomic and human papillomavirus profiling of an oral cancer cohort identifies TP53 as a predictor of overall survival. Cancers Head Neck 4:5, 2019
2. Ghasemi F, Black M, Sun R X, et al: High-throughput testing in head and neck squamous cell carcinoma identifies agents with preferential activity in human papillomavirus-positive or negative cell lines. Oncotarget 9:26064-26071, 2018
3. Dobin A, Davis C A, Schlesinger F, et al: STAR: ultrafast universal RNA-seq aligner. Bioinformatics 29:15-21, 2013
4. Anders S, Pyl P T, Huber W: HTSeq—a Python framework to work with high-throughput sequencing data. Bioinformatics 31:166-9, 2015
5. Love M I, Huber W, Anders S: Moderated estimation of fold change and dispersion for RNA-seq data with DESeq2. Genome Biol 15:550, 2014
6. Cancer Genome Atlas N: Comprehensive genomic characterization of head and neck squamous cell carcinomas. Nature 517:576-82, 2015
7. Liu X, Liu P, Chernock R D, et al: Impact of human papillomavirus on the tumor microenvironment in oropharyngeal squamous cell carcinoma. Int J Cancer, 2021
8. Cavalieri S, Serafini M S, Carenzo A, et al: Clinical Validity of a Prognostic Gene Expression Cluster-Based Model in Human Papillomavirus-Positive Oropharyngeal Carcinoma. JCO Precision Oncology:1666-1676, 2021
9. Lu X J D, Liu K Y P, Prisman E, et al: Prognostic value and cost benefit of HPV testing for oropharyngeal cancer patients. Oral Diseases, 2021
10. Kelley D Z, Flam E L, Izumchenko E, et al: Integrated Analysis of Whole-Genome ChIP-Seq and RNA-Seq Data of Primary Head and Neck Tumor Samples Associates HPV Integration Sites with Open Chromatin Marks. Cancer Res 77:6538-6550, 2017
11. Hao Y, Yang L, Galvao Neto A, et al: HPViewer: sensitive and specific genotyping of human papillomavirus in metagenomic DNA. Bioinformatics 34:1986-1995, 2018
12. Becht E, Giraldo N A, Lacroix L, et al: Estimating the population abundance of tissue-infiltrating immune and stromal cell populations using gene expression. Genome Biol 17:218, 2016
13. Sturm G, Finotello F, Petitprez F, et al: Comprehensive evaluation of transcriptome-based cell-type quantification methods for immuno-oncology. Bioinformatics 35:i436-i445, 2019
14. Gu Z, Eils R, Schlesner M: Complex heatmaps reveal patterns and correlations in multidimensional genomic data. Bioinformatics 32:2847-9, 2016
15. Petitprez F, de Reynies A, Keung E Z, et al: B cells are associated with survival and immunotherapy response in sarcoma. Nature 577:556-560, 2020
16. Huang C, Chen L, Savage S R, et al: Proteogenomic insights into the biology and treatment of HPV-negative head and neck squamous cell carcinoma. Cancer Cell 39:361-379 e16, 2021
17. Hobbs A J, Brockton N T, Matthews T W, et al: Primary treatment for oropharyngeal squamous cell carcinoma in Alberta, Canada: A population-based study. Head Neck 39:2187-2199, 2017
18. Bankhead P, Loughrey M B, Fernandez J A, et al: QuPath: Open source software for digital pathology image analysis. Sci Rep 7:16878, 2017 An online UWO3 calculator is available (https://www.nicholslab.com/uwo3calculator).

### Statistical Analysis

All statistical analyses were performed with R software (v4.0.5) using the following packages: survival (v3.2.10), ggpubr (v0.4.0), stats (v4.0.5), and rms (v6.2-0). Wilcoxon rank sum test (2 categories) and Kruskal-Wallis test (>2 categories) were used to examine all relationships between categorical variables and quantitative variables. P-341 values were corrected for multiple testing using the Benjamini-Hochberg method. Fisher's method was used to combine Wilcoxon rank sum test P-values across cohorts. The Fisher's Exact test was used to analyze contingency tables. Survival analyses were analyzed with the log-rank test and Cox proportional hazards model with the survival (v3.1-12) package. The proportional hazards assumption was assessed using Schoenfeld residuals. Hazard ratio in the JHU cohort was estimated using Cox Regression with Firth's penalized likelihood⁵⁵, implemented using the coxphf package (v1.13.1), as monotone likelihood is observed due to no events in the immune rich group. Meta-analysis of the association between UWO3 immune class and disease-free survival using the inverse variance method through the R package meta (v5.1-1) using log 10 HR and SEM in a fixed-effect model. The significance of any discrepancies in the estimates of the treatment effects from the different cohorts was assessed using Cochrane's test for heterogeneity and the I2 statistic as described previously⁵⁶. Heterogeneity was considered statistically significant if the P value was less than 0.10 for the χ2 test. Brier's error analysis of Cox models was calculated using the package pec (v2021.10.11). The relative importance of each parameter to survival risk was assessed using the χ² from R package rms (v6.2-0). All tests were 2-sided.

### Results

### Development and Validation of the UWO3 Immune Classification

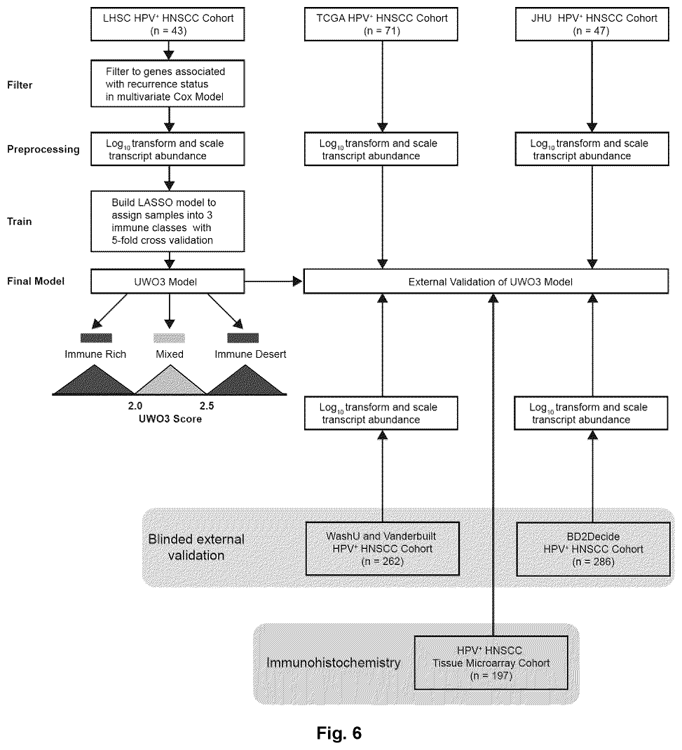

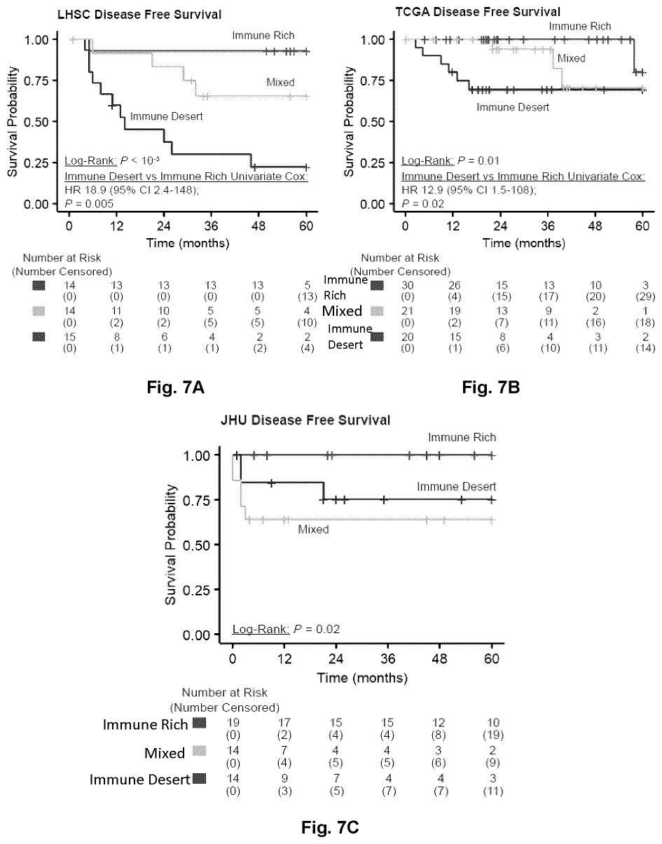

To reveal transcriptomic features predictive of treatment response, we performed RNA-seq on 43 HPV⁺ 156 HNSCC tumours, 16 of which experienced local, regional, or distant recurrence. As specific tumour microenvironment alterations are associated with recurrence in HPV⁺ 157 HNSCC²⁴⁻²⁷, we characterized the tumour microenvironment (TME) by the abundance of distinct cell populations²⁸. Through unsupervised clustering of these estimated immune cell abundances (see Methods), we classified samples into three categories: “immune rich”, “immune desert”, and “mixed” (FIG. 5A). These three TME subtypes exhibited distinct patterns of overall survival (OS; P=0.003, log rank test; FIG. 5B) and disease-free survival (DFS; P<10-3, log rank test; FIG. 5C). To facilitate translation of these results into the clinic, we developed a minimal classifier based on using the Least Absolute Shrinkage and Selection Operator (LASSO; FIG. 6) to stratify patients into one of three immune classes. The resulting classifier, referred to as University of Western Ontario 3 (UWO3), is based on the abundance of three transcripts (CD3E, IRF4, and ZAP70) and assigns immune classes strongly associated with DFS in our discovery cohort, as expected (P<10⁻³, log rank test, FIG. 7A).

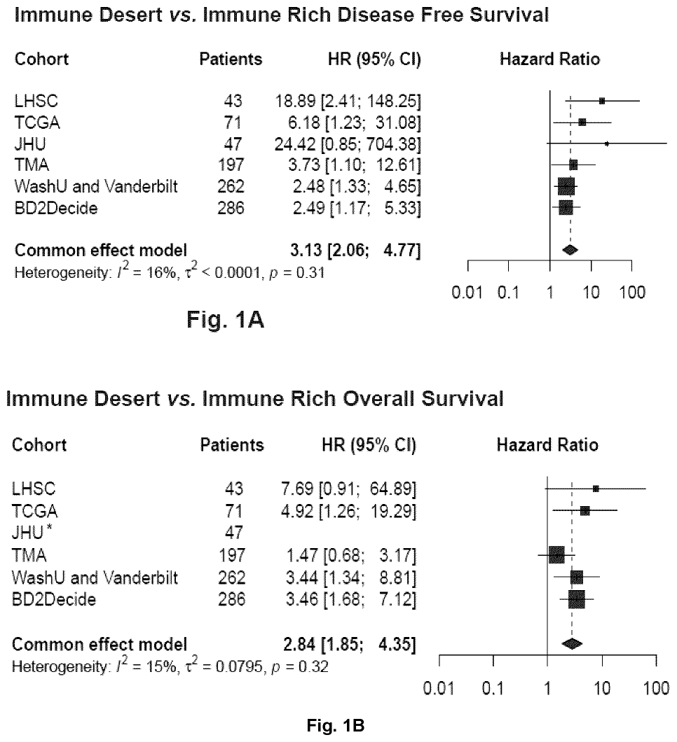

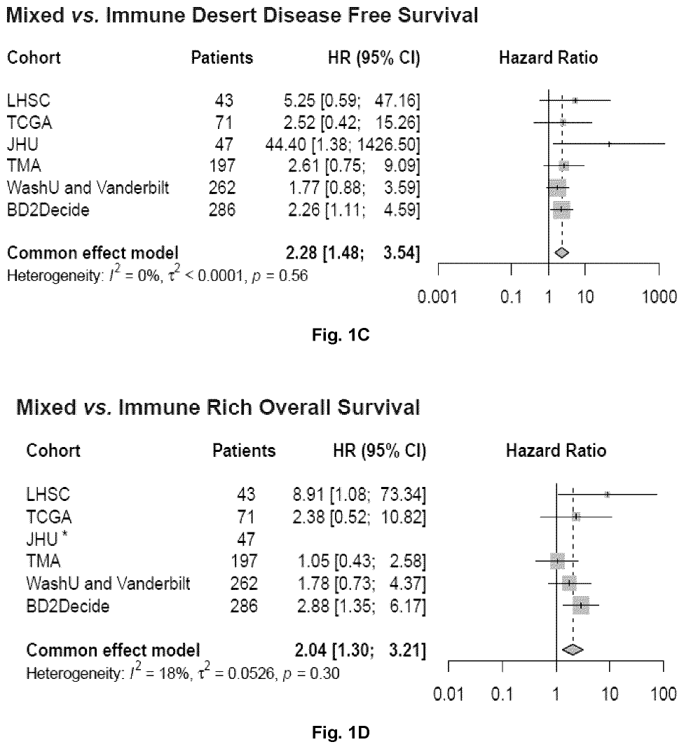

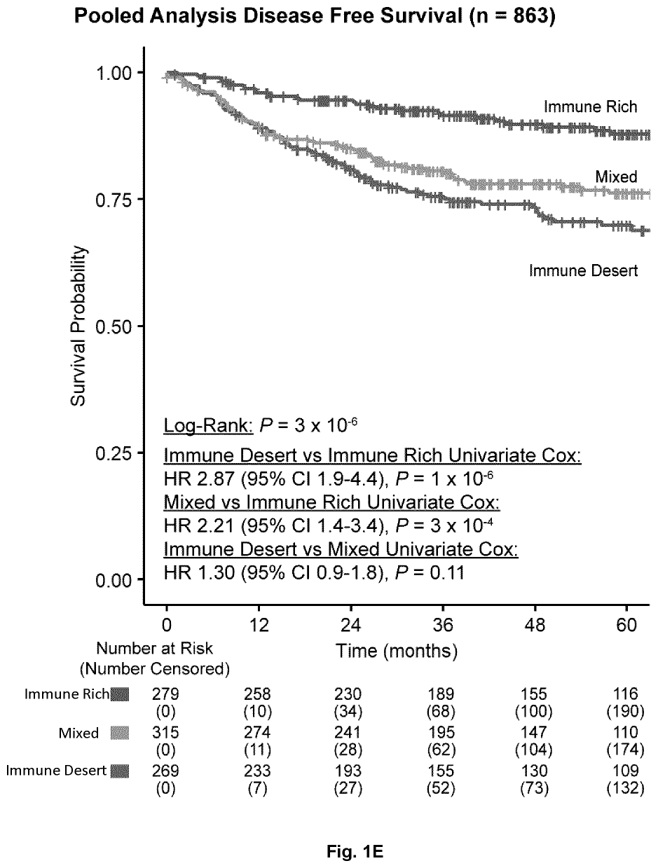

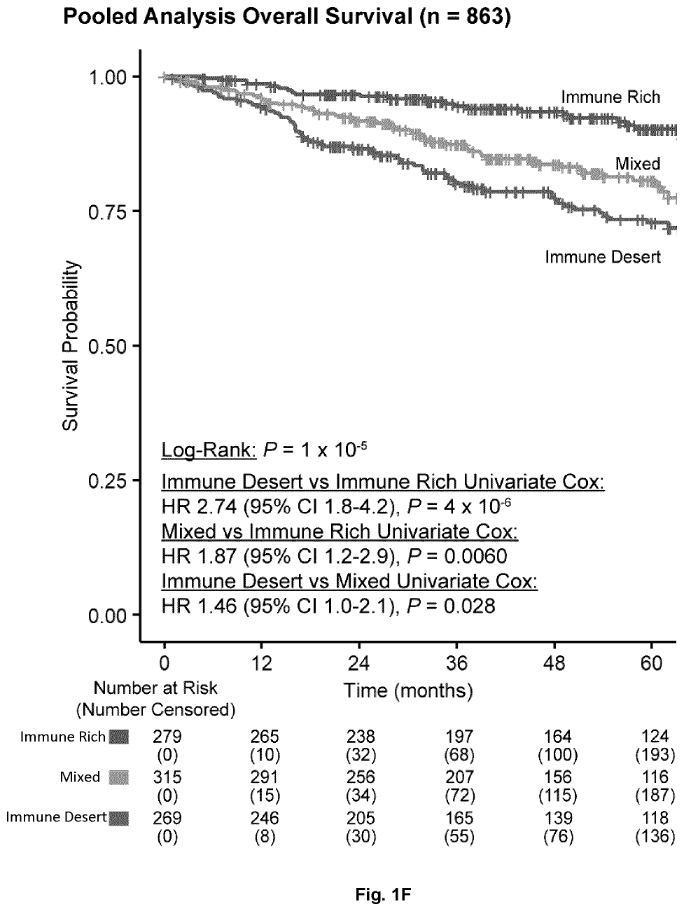

We next tested five independent cohorts to validate the association between UWO3 immune class and survival outcomes in HPV⁺ HNSCC. We first used the public The Cancer Genome Atlas (TCGA)²⁹ (n=71) and Johns Hopkins University (JHU; n=47) HPV⁺ HNSCC RNA-seq cohorts³⁰. In both TCGA and JHU, the immune rich patients had improved DFS and OS (TCGA: P=0.01, JHU: P=0.02, log rank test, FIGS. 1A, 1C, FIGS. 7B and 7C).

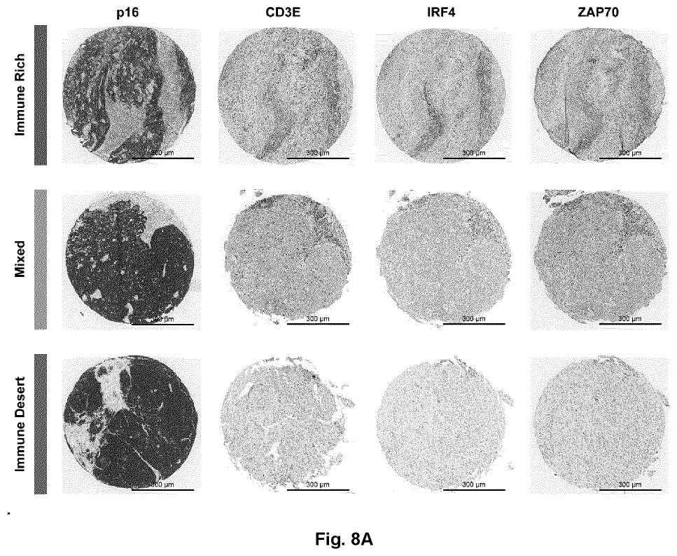

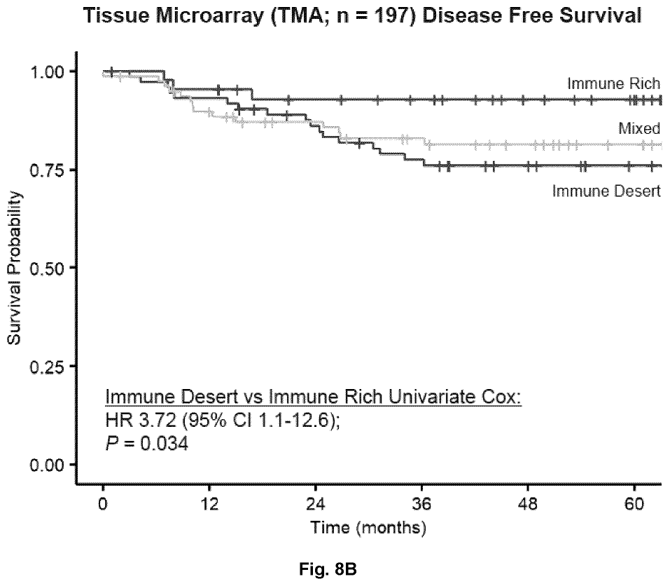

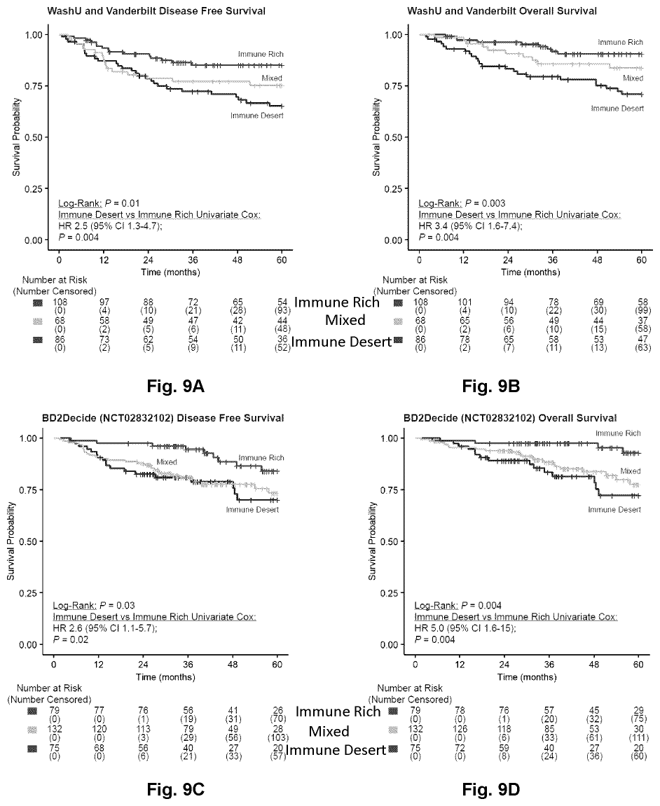

Next, we employed clinically-validated antibodies for the proteins corresponding to the transcripts within the UWO3 score (CD3E, ZAP70, and IRF4) that are used routinely in clinical pathology labs for hematologic malignancies. As immunohistochemistry (IHC) is cost-effective and broadly available, we applied the UWO3 score to a tissue microarray (TMA) consisting of 197 independent HPV⁺ HNSCC patients (FIG. 8A). Immune desert patients experienced inferior DFS (HR=3.1, 95% Cl 1.1-9.7, P=0.038, univariate Cox model; FIG. 1A, FIG. 8B) compared to the immune rich patients, highlighting the potential of the UWO3 immune classification as to be implemented as an IHC assay.

Finally, to validate the association between UWO3 score and survival outcomes, we performed blinded validations in a retrospective cohort and a prospective cohort. Patients were assigned to immune classes using UWO3 before unmasking of clinical outcomes by our external collaborators. In a retrospective cohort of HPV⁺ HNSCC patients (n=262) treated primarily with surgery at Washington University at St. Louis (WashU) and Vanderbilt University³¹, the immune classes exhibited distinct DFS (P=0.01, log rank test; immune desert vs. immune rich: HR=2.5, 95% Cl 1.3-4.7, P=0.004, univariate Cox model; FIGS. 1A, 1C, FIG. 9A) and overall survival (OS) (P=0.01, log rank test; immune desert vs. immune rich: HR=3.44, 95% Cl 1.59-7.44, P=0.002, univariate Cox model; FIGS. 1B, 1D, FIG. 9B). In the prospective Big Data and Models for Personalized Head and Neck Cancer Decision Support (BD2Decide) study (n=286, NCT02832102) of 286 locoregionally-advanced p16-positive patients treated homogeneously with radiation and/or chemotherapy at seven European institutions³², the immune classes exhibited distinct DFS (P=0.03, log rank test; immune desert vs. immune rich: HR=2.6, 95% Cl 1.1-5.7, P=0.02, univariate Cox model; FIG. 1A, 1C, FIG. 9C) and OS (P=0.004, log rank test; immune desert vs. immune rich: HR=5.0, 95% Cl 1.6-15, P=0.004, univariate Cox model; FIGS. 1B, 1D, FIG. 9D). Taken together, we have shown that the UWO3 immune class is robustly associated with survival outcomes in six independent cohorts across different profiling platforms and geographic jurisdictions.

### Pooled Analysis of all Cohorts Underscores UWO3 Immune Class as a Strong Independent Predictor

As the association between immune groups and survival outcomes for each cohort were homogeneous between cohorts (I²<50%, P>0.10; FIGS. 1A-D), we performed a pooled analysis of all six cohorts (LHSC, TCGA, JHU, TMA, WashU, and Vanderbilt, and BD2Decide; total n=906). The immune class defined by UWO3 was strongly associated with DFS (P=4×10⁻⁸, log rank test, FIG. 1E) and OS (P=9×10-7, log rank test, FIG. 1F) in this pooled cohort. The 5-year disease-free survival (DFS) probabilities for the immune rich, mixed, and immune deserts group were 88.1%, 75.7%, and 67.3%, respectively. Immune desert patients exhibited inferior DFS over the immune rich (HR=3.23, 95% Cl 2.1-4.9, P=2×10-8, univariate Cox model; FIG. 1E) and mixed (HR=1.38, 95% Cl 1.0-1.9, P=0.041, univariate Cox model; FIG. 1E) patients. The mixed patients also exhibited worse DFS over the immune rich patients (HR=2.34, 95% Cl 1.5-3.6, P=6×10⁻⁵, univariate Cox model; FIG. 1E). The 5-year OS probabilities for the immune rich, mixed, and immune deserts group were 90.4%, 79.3%, and 71.8%, respectively. Immune desert patients exhibited inferior OS over the immune rich (HR=2.89, 95% Cl 1.9-4.4, P=7×10⁻⁷, univariate Cox model; FIG. 1F) and mixed (HR=1.40, 95% Cl 1.0-1.9, P=0.041, FIG. 1F) patients. The patients with mixed tumours also exhibited worse OS than immune rich patients (HR=2.05, 95% Cl 1.3-3.2, P=0.0011, univariate Cox model; FIG. 1F). The survival differences by UWO3 immune group persisted for patients undergoing both primary surgery (n=324. Immune rich vs. immune desert DFS: HR=3.12, 95% Cl 1.7-5.9, P=0.0004, univariate Cox model;

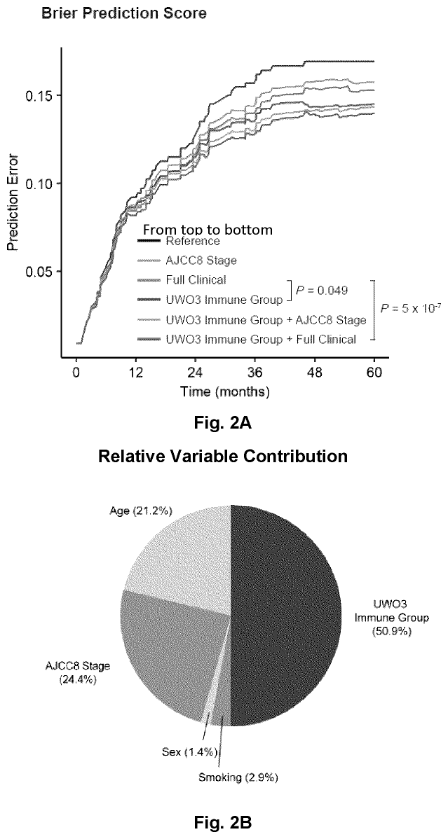

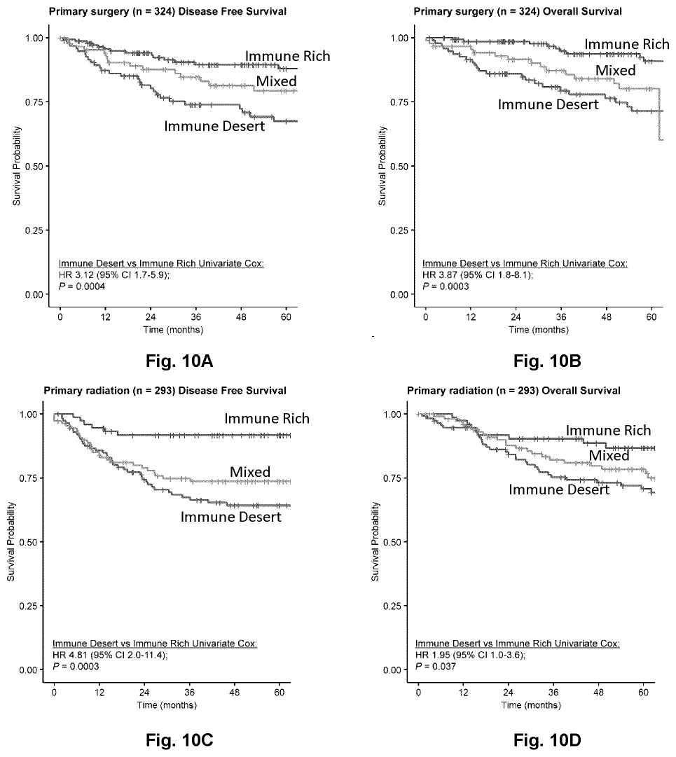

FIGS. 10A-10B) or primary radiation (n=293. Immune rich vs. immune desert DFS: HR=4.81, 95% Cl 2.0-11.4, P=0.0003, univariate Cox model; FIGS. 10C-10D). In a multivariate Cox proportional hazards model stratified for cohort, the association between the UWO3 immune class and DFS was independent of other clinical factors (Immune desert vs. immune rich: HR=9.0, 95% Cl 3.17-25.5, P=3.6×10-5; Mixed vs. immune rich: HR=6.4 (95% Cl 2.2-18.7, P=0.0006; Table 3). Brier score analysis demonstrates that the UWO3 immune group alone had a lower prediction error for disease-free survival than a Cox model of clinical factors (AJCC8 stage, sex, smoking status, and age) (P=0.049, χ² test; FIG. 2A). Furthermore, integration of the UWO3 immune group with other clinical factors (UWO3+Full Clinical) further decreased prediction error (P=5×10-7, χ² test; FIG. 2A). We analyzed the relative contribution of each parameter to predict DFS and identified UWO3 immune group (50.9%, FIG. 2B) as the strongest parameter, compared to other clinical factors. Thus, UWO3 immune class is a strong independent prognostic factor that can improve the risk-stratification of HPV⁺ HNSCC.

### UWO3 Immune Classification has Implications for Immunotherapy and Treatment De-Intensification

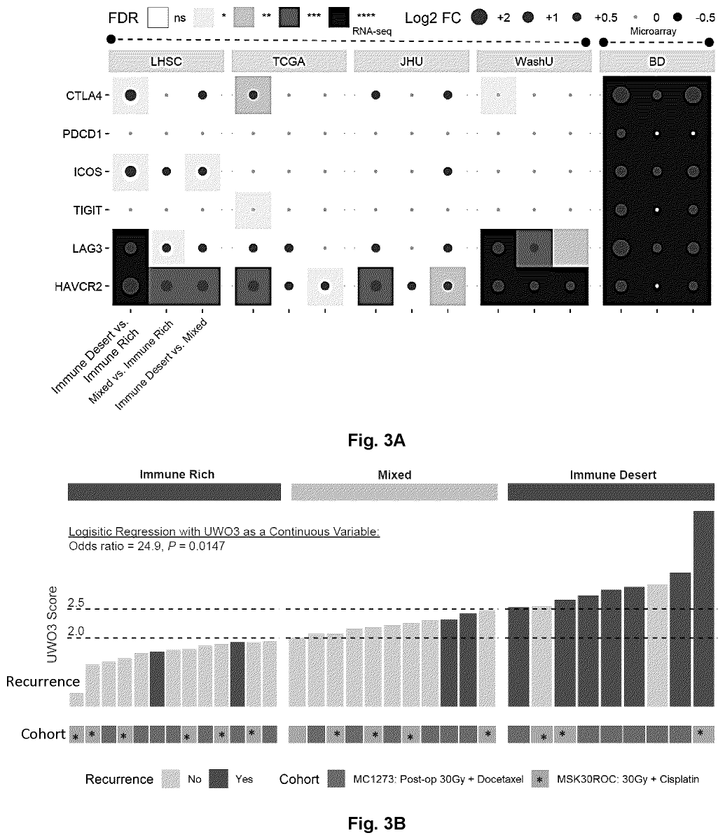

The poor survival outcomes of the immune desert and mixed groups compared to the immune rich group suggest immunostimulatory approaches may improve outcomes for these two groups of patients. Thus, we aimed to assess whether each immune class exhibit distinct abundance of immune checkpoints and focused on the immune co-receptors expressed on T-cells CTLA4, PDCD1 (encodes PD1), ICOS, TIGIT, LAG3, and HAVCR2 (encodes TIM3)³³. After normalizing for T-cell abundance, we compared RNA abundance between the immune groups. The immune desert group expressed higher normalized abundance of HAVCR2 (encodes TIM3) compared to the immune rich in all five cohorts (FDR<4.4×10-52, Fisher's Method, FIG. 3A). These results support the exploration of anti-TIM3 antibodies and/or other immunostimulatory strategies in high-risk patients. For example, these patients may be ideal candidates for treatment with anti-PD1 antibodies (such as nivolumab), anti-PDL1 antibodies (such as pembrolizumab), experimental anti-TIM3 antibodies (such as sabatolimab), or combinations of these treatments.

As the immune rich patients exhibit excellent survival outcomes, we hypothesized that the UWO3 score can identify patients who respond favourably to de-intensified treatment. We used RNA seq data from the phase II MC1273 (NCT01932697) trial¹⁷, which tested an aggressive de-escalation regimen of 30 Gy radiation with concurrent docetaxel post-surgery, and the 30ROC trial (NCT00606294)³⁴, in which patients received 30 Gy radiation and cisplatin. Higher UWO3 score was associated with higher odds of recurrence following aggressive treatment de-escalation (Odds ratio: 24.9, P=0.0147, logistic regression with UWO3 as continuous variable stratified for cohort, FIG. 3B). Strikingly, 7 out of 9 patients (77.8%, FIG. 3B) in the immune desert group developed recurrence, while only 4 out of the 24 patients (16.6%) in the immune rich and mixed recurred.

Although HPV⁺ HNSCC patients have improved prognosis over their HPV-negative counterparts, a significant portion of patients still recur after initial treatment and are at risk of death. The present disclosure demonstrates that the pre-treatment TME has dramatic effects in determining the prognosis of HPV⁺ HNSCC patients. Provided herein is a clinically translatable, extensively validated UWO3 immune classification tool that allows biomarker-driven individualized treatment in HPV⁺ HNSCC (FIG. 4).

The immune classification presented herein is based on the expression levels or abundance of CD3E, ZAP70 and IRF4. CD3E is part of the T-cell receptor complex and its down-regulation on T-cells has been linked to worse prognosis in HNSCC³⁵. ZAP70 plays important role in T-cell receptor signaling but is also highly expressed on NK-cells³⁶. IRF4 directs the development, affinity maturation, and terminal differentiation of B cells, but also plays important roles in monocyte differentiation³⁷⁻⁴¹.

Patients with immune rich pre-treatment tumours have improved prognosis compared to the mixed and immune desert groups consistently across six different cohorts regardless of treatment. Thus, patients who are immune rich and have favorable clinical factors may be ideal candidates for aggressive treatment de-intensification. The inflammatory TME of the immune rich group and dense infiltration of PD1⁺ CD8 T cells pre-treatment supports further exploration of substitution of chemotherapy with immunotherapy in de-escalation settings. The three-arm phase Ill randomized controlled trial NRG-HN005 (NCT03952585) is currently evaluating such a regimen (60 Gy plus nivolumab) against the standard treatment of 70 Gy with cisplatin and a de-intensified regimen of 60 Gy plus cisplatin. Furthermore, the association between patients who have immune desert pre-treatment tumours and poor survival outcomes supports the investigation of neoadjuvant immunotherapy approaches, which have been found to increase T-cell density within HNSCC tumour⁴².

The key limitation of our study is that the treatment protocols delivered in each cohort were not uniform. However, the strong association of UWO3 with DFS in six cohorts, which spans different geographic jurisdictions, treatment methods, and patient populations suggests that the immune classes can treatment-agnostically predict survival outcomes. Another limitation is that our analysis of immune co-receptors abundance was performed using bulk RNA profiling and thus cannot distinguish the specific cell types expressing these immune checkpoint receptors. Moreover, some of these molecules (such as TIM3) are expressed on T-cells as well as dendritic cells and macrophages^{33,43,44}. Nevertheless, recent work in HPV⁺ HNSCC using single-cell RNA-seq demonstrated that T-cells make up the majority of immune cell populations within the TME and are the major contributors of these molecules^{24,25}. As single-cell technologies become more accessible, cohort-level studies to fully dissect HPV⁺ HNSCC and characteristics associated with survival will undoubtedly pave the way for a more nuanced understanding of HPV⁺ HNSCC TME.

The ideal patient population for treatment de-intensification and intensification in HPVP⁺ HNSCC remains controversial¹³. Our retrospectively and prospectively-validated immune classification provides a readily implemented means to identify patients that may maximally benefit from treatment de-escalation or intensification. We are in the process of testing these hypotheses through biomarker-driven randomized controlled de-intensification and intensification trials.

### REFERENCES

1. Menezes F D S, Fernandes G A, Antunes J L F, et al: Global incidence trends in head and neck cancer for HPV-related and -unrelated subsites: A systematic review of population-based studies. Oral Oncol 115:105177, 2021
2. Cramer J D, Burtness B, Le Q T, et al: The changing therapeutic landscape of head and neck cancer. Nature Reviews Clinical Oncology 16:669-683, 2019
3. Ang K K, Harris J, Wheeler R, et al: Human papillomavirus and survival of patients with oropharyngeal cancer. N Engl J Med 363:24-35, 2010
4. Gillison M L, Chaturvedi A K, Anderson W F, et al: Epidemiology of Human Papillomavirus-Positive Head and Neck Squamous Cell Carcinoma. J Clin Oncol 33:3235-42, 2015
5. Quon H, Vapiwala N, Forastiere A, et al: Radiation Therapy for Oropharyngeal Squamous Cell Carcinoma: American Society of Clinical Oncology Endorsement of the American Society for Radiation Oncology Evidence-Based Clinical Practice Guideline. J Clin Oncol 35:4078-4090, 2017
6. Pfister D G, Spencer S, Adelstein D, et al: Head and Neck Cancers, Version 2.2020, NCCN Clinical Practice Guidelines in Oncology. J Natl Compr Canc Netw 18:873-898, 2020
7. O'Sullivan B, Huang S H, Siu L L, et al: Deintensification candidate subgroups in human papillomavirus-related oropharyngeal cancer according to minimal risk of distant metastasis. J Clin Oncol 31:543-50, 2013
8. Kelly J R, Husain Z A, Burtness B: Treatment de-intensification strategies for head and neck cancer. Eur J Cancer 68:125-133, 2016
9. Price K A R, Nichols A C, Shen C J, et al: Novel Strategies to Effectively De-escalate Curative-Intent Therapy for Patients With HPV-Associated Oropharyngeal Cancer: Current and Future Directions. Am Soc Clin Oncol Educ Book 40:1-13, 2020
10. Leeman J E, Li J G, Pei X, et al: Patterns of Treatment Failure and Postrecurrence Outcomes Among Patients With Locally Advanced Head and Neck Squamous Cell Carcinoma After Chemoradiotherapy Using Modern Radiation Techniques. JAMA Oncol 3:1487-1494, 2017
11. Gillison M L, Trotti A M, Harris J, et al: Radiotherapy plus cetuximab or cisplatin in human papillomavirus-positive oropharyngeal cancer (NRG Oncology RTOG 1016): a randomised, multicentre, non-inferiority trial. Lancet 393:40-50, 2019
12. Mehanna H, Robinson M, Hartley A, et al: Radiotherapy plus cisplatin or cetuximab in low-risk human papillomavirus-positive oropharyngeal cancer (De-ESCALaTE HPV): an open-label randomised controlled phase 3 trial. Lancet 393:51-60, 2019
13. Adelstein D J, Ismaila N, Ku J A, et al: Role of Treatment Deintensification in the Management of p16+ Oropharyngeal Cancer: ASCO Provisional Clinical Opinion. J Clin Oncol 37:1578-1589, 2019
14. Mehanna H, Rischin D, Wong S J, et al: De-Escalation After DE-ESCALATE and RTOG 1016: A Head and Neck Cancer InterGroup Framework for Future De-Escalation Studies. J Clin Oncol 38:2552-2557, 2020
15. Cmelak A J, Ferris R L, Chen A M, et al: Treatment De-intensification for HPV-Positive Oropharynx Cancer: What Is Currently Acceptable? J Clin Oncol 39:2732-2733, 2021
16. Fakhry C, Zhang Q, Nguyen-Tan P F, et al: Development and Validation of Nomograms Predictive of Overall and Progression-Free Survival in Patients With Oropharyngeal Cancer. J Clin Oncol 35:4057-4065, 2017
17. Ma D J, Price K A, Moore E J, et al: Phase II Evaluation of Aggressive Dose De-Escalation for Adjuvant Chemoradiotherapy in Human Papillomavirus-Associated Oropharynx Squamous Cell Carcinoma. J Clin Oncol 37:1909-1918, 2019
18. Yom S S, Torres-Saavedra P, Caudell J J, et al: Reduced-Dose Radiation Therapy for HPV-Associated Oropharyngeal Carcinoma (NRG Oncology HN002). J Clin Oncol 39:956-965, 2021
19. Morris L G T, Chandramohan R, West L, et al: The Molecular Landscape of Recurrent and Metastatic Head and Neck Cancers: Insights From a Precision Oncology Sequencing Platform. JAMA Oncol 3:244-255, 2017
20. Lee N, Schoder H, Beattie B, et al: Strategy of Using Intratreatment Hypoxia Imaging to Selectively and Safely Guide Radiation Dose De-escalation Concurrent With Chemotherapy for Locoregionally Advanced Human Papillomavirus-Related Oropharyngeal Carcinoma. Int J Radiat Oncol Biol Phys 96:9-17, 2016
21. Zhang Y, Koneva L A, Virani S, et al: Subtypes of HPV-Positive Head and Neck Cancers Are Associated with HPV Characteristics, Copy Number Alterations, PIK3CA Mutation, and Pathway Signatures. Clin Cancer Res 22:4735-45, 2016
22. Gross A M, Orosco R K, Shen J P, et al: Multi-tiered genomic analysis of head and neck cancer ties TP53 mutation to 3p loss. Nat Genet 46:939-43, 2014
23. Gleber-Netto F O, Rao X, Guo T, et al: Variations in HPV function are associated with survival in squamous cell carcinoma. JCI Insight 4:e124762, 2019
24. Cillo A R, Kurten C H L, Tabib T, et al: Immune Landscape of Viral- and Carcinogen-Driven Head and Neck Cancer. Immunity 52:183-199 e9, 2020
25. Lechner A, Schlosser H A, Thelen M, et al: Tumor-associated B cells and humoral immune response in head and neck squamous cell carcinoma. Oncoimmunology 8:1535293, 2019
26. Nelson H H, Pawlita M, Michaud D S, et al: Immune Response to HPV16 E6 and E7 Proteins and Patient Outcomes in Head and Neck Cancer. JAMA Oncol 3:178-185, 2017
27. Petitprez F, de Reynies A, Keung E Z, et al: B cells are associated with survival and immunotherapy response in sarcoma. Nature 577:556-560, 2020
28. Becht E, Giraldo N A, Lacroix L, et al: Estimating the population abundance of tissue-infiltrating immune and stromal cell populations using gene expression. Genome Biol 17:218, 2016
29. Cancer Genome Atlas Research N, Weinstein J N, Collisson E A, et al: The Cancer Genome Atlas Pan-Cancer analysis project. Nat Genet 45:1113-20, 2013
30. Kelley D Z, Flam E L, Izumchenko E, et al: Integrated Analysis of Whole-Genome ChIP-Seq and RNA-Seq Data of Primary Head and Neck Tumor Samples Associates HPV Integration Sites with Open Chromatin Marks. Cancer Res 77:6538-6550, 2017
31. Liu X, Liu P, Chernock R D, et al: Impact of human papillomavirus on the tumor microenvironment in oropharyngeal squamous cell carcinoma. Int J Cancer, 2021
32. Cavalieri S, Serafini M S, Carenzo A, et al: Clinical Validity of a Prognostic Gene Expression Cluster-Based Model in Human Papillomavirus-Positive Oropharyngeal Carcinoma. JCO Precision Oncology:1666-1676, 2021
33. Anderson A C, Joller N, Kuchroo V K: Lag-3, Tim-3, and TIGIT: Co-inhibitory Receptors with Specialized Functions in Immune Regulation. Immunity 44:989-1004, 2016
34. Riaz N, Sherman E, Pei X, et al: Precision Radiotherapy: Reduction in Radiation for Oropharyngeal Cancer in the 30 ROC Trial. J Natl Cancer Inst 113:742-751, 2021
35. Upreti D, Zhang M L, Bykova E, et al: Change in CD3zeta-chain expression is an independent predictor of disease status in head and neck cancer patients. Int J Cancer 139:122-9, 2016
36. Colucci F, Schweighoffer E, Tomasello E, et al: Natural cytotoxicity uncoupled from the Syk and ZAP-70 intracellular kinases. Nat Immunol 3:288-94, 2002
37. Klein U, Casola S, Cattoretti G, et al: Transcription factor IRF4 controls plasma cell differentiation and class-switch recombination. Nat Immunol 7:773-82, 2006
38. Klammt C, Novotna L, Li D T, et al: T cell receptor dwell times control the kinase activity of Zap70. Nat Immunol 16:961-9, 2015
39. Gu Y, Chae H D, Siefring J E, et al: RhoH GTPase recruits and activates Zap70 required for T cell receptor signaling and thymocyte development. Nat Immunol 7:1182-90, 2006
40. Hagman J: Critical Functions of IRF4 in B and T Lymphocytes. J Immunol 199:3715-3716, 2017
41. Lehtonen A, Veckman V, Nikula T, et al: Differential expression of IFN regulatory factor4 gene in human monocyte-derived dendritic cells and macrophages. J Immunol 175:6570-9, 2005
42. Ferrarotto R, Bell D, Rubin M L, et al: Impact of Neoadjuvant Durvalumab with or without Tremelimumab on CD8(+) Tumor Lymphocyte Density, Safety, and Efficacy in Patients with Oropharynx Cancer: CIAO Trial Results. Clin Cancer Res 26:3211-3219, 2020
43. Dixon K O, Tabaka M, Schramm M A, et al: TIM-3 restrains anti-tumour immunity by regulating inflammasome activation. Nature 595:101-106, 2021
44. Shayan G, Srivastava R, Li J, et al: Adaptive resistance to anti-PD1 therapy by Tim-3 upregulation is mediated by the PI3K-Akt pathway in head and neck cancer. OncoImmunology 6:e1261779, 2017
45. Kurten C H L, Kulkarni A, Cillo A R, et al: Investigating immune and non-immune cell interactions in head and neck tumors by single-cell RNA sequencing. Nat Commun 12:7338, 2021
46. Cancer Genome Atlas N: Comprehensive genomic characterization of head and neck squamous cell carcinomas. Nature 517:576-82, 2015
47. Lu X J D, Liu K Y P, Prisman E, et al: Prognostic value and cost benefit of HPV testing for oropharyngeal cancer patients. Oral Diseases, 2021
48. Altman D G, McShane L M, Sauerbrei W, et al: Reporting Recommendations for Tumor Marker Prognostic Studies (REMARK): explanation and elaboration. PLoS Med 9:e1001216, 2012
49. Heinze G, Schemper M: A solution to the problem of monotone likelihood in Cox regression. Biometrics 57-114-9, 2001
50. Higgins J P, Thompson S G, Deeks J J, et al: Measuring inconsistency in meta-analyses. BMJ 327-557-60, 2003

TABLE 1
Detailed clinical information in the LHSC cohort

| | | | | | | Recurrence | | | |
|---|---|---|---|---|---|---|---|---|---|
| | Number of | | Gender | | Smoking | (0 = Disease | Disease- | Death | |
| Sample | RNA-seq | HPV | (1 = male, | Age at | (pack- | free, 1 = | free | (0 = alive, | Overall |
| ID | Reads | Subtype | 2 = female) | Diagnosis | years) | Recurrence) | survival | 1 = death) | survival |
| 38 | 65999107 | HPV16 | 1 | 53 | 40 | 0 | 60 | 0 | 60 |
| 54 | 61547655 | HPV16 | 1 | 65 | 5 | 0 | 60 | 0 | 60 |
| 56 | 64428876 | HPV16 | 2 | 57 | 0 | 0 | 60 | 0 | 60 |
| 58 | 65641071 | HPV16 | 1 | 54 | 40 | 0 | 1 | 0 | 42 |
| 109 | 60466556 | HPV16 | 1 | 84 | 0 | 1 | 4 | 1 | 5 |
| 113 | 68214916 | HPV16 | 1 | 60 | 35 | 1 | 24 | 1 | 31 |
| 119 | 81397202 | HPV16 | 2 | 42 | 14 | 0 | 31 | 0 | 31 |
| 122 | 77856158 | HPV16 | 1 | 62 | 0 | 0 | 35 | 1 | 35 |
| 123 | 66738329 | HPV16 | 1 | 48 | 25 | 0 | 60 | 0 | 60 |
| 134 | 76865871 | HPV16 | 1 | 43 | 14 | 1 | 46 | 0 | 60 |
| 139 | 57525948 | HPV16 | 1 | 57 | 15 | 0 | 60 | 0 | 60 |
| 140 | 61083732 | HPV16 | 1 | 69 | 0 | 0 | 60 | 0 | 60 |
| 146 | 61673936 | HPV16 | 1 | 49 | 0 | 0 | 57 | 0 | 57 |
| 159 | 51582250 | HPV16 | 2 | 69 | 50 | 1 | 32 | 1 | 50 |
| 160 | 62505206 | HPV16 | 1 | 62 | 0 | 0 | 56 | 0 | 56 |
| 162 | 71680973 | HPV16 | 1 | 45 | 0 | 0 | 60 | 0 | 60 |
| 168 | 69451492 | HPV16 | 1 | 54 | 0 | 0 | 52 | 0 | 52 |
| 176 | 65155113 | HPV16 | 1 | 72 | 50 | 1 | 5 | 1 | 9 |
| 194 | 72681370 | HPV16 | 1 | 72 | 0 | 1 | 29 | 1 | 29 |
| 195 | 59299274 | HPV35 | 1 | 52 | 0 | 0 | 60 | 0 | 60 |
| 200 | 86544489 | HPV16 | 2 | 55 | 30 | 1 | 21 | 1 | 26 |
| 204 | 75625870 | HPV16 | 1 | 57 | 40 | 0 | 56 | 0 | 56 |
| 214 | 78312700 | HPV16 | 1 | 80 | 60 | 1 | 5 | 1 | 6 |
| 230 | 69788662 | HPV16 | 1 | 58 | 0 | 0 | 1 | 1 | 1 |
| 232 | 61468783 | HPV16 | 1 | 63 | 0 | 1 | 14 | 0 | 60 |
| 255 | 71296963 | HPV16 | 1 | 71 | 25 | 0 | 60 | 0 | 60 |
| 265 | 83464602 | HPV16 | 1 | 67 | 57 | 0 | 11 | 0 | 11 |
| 266 | 71103390 | HPV16 | 1 | 46 | 30 | 0 | 56 | 0 | 56 |
| 304 | 60058858 | HPV16 | 1 | 64 | 0 | 0 | 52 | 0 | 52 |
| 305 | 58554788 | HPV16 | 1 | 75 | 0 | 0 | 60 | 0 | 60 |
| 323 | 99087094 | HPV16 | 2 | 56 | 25 | 1 | 6 | 0 | 39 |
| 358 | 66042403 | HPV16 | 1 | 76 | 30 | 0 | 55 | 0 | 55 |
| 365 | 61968534 | HPV16 | 1 | 56 | 7 | 0 | 55 | 0 | 55 |
| 376 | 58176411 | HPV16 | 1 | 53 | 0 | 0 | 50 | 0 | 50 |
| 381 | 56398318 | HPV16 | 1 | 66 | 15 | 1 | 26 | 1 | 26 |
| 402 | 64187649 | HPV16 | 1 | 73 | 25 | 0 | 47 | 0 | 47 |
| 440 | 63832387 | HPV16 | 1 | 63 | 40 | 1 | 8 | 0 | 41 |
| 471 | 67096036 | HPV16 | 1 | 54 | 0 | 0 | 34 | 0 | 34 |
| 485 | 62123246 | HPV16 | 1 | 64 | 0 | 1 | 13 | 1 | 31 |
| 510 | 71604963 | HPV16 | 1 | 72 | 20 | 1 | 11 | 1 | 24 |
| 571 | 73330879 | HPV16 | 1 | 68 | 0 | 1 | 6 | 1 | 16 |
| 619 | 69130717 | HPV16 | 1 | 60 | 0 | 1 | 5 | 0 | 13 |
| 624 | 74053278 | HPV16 | 1 | 57 | 15 | 0 | 60 | 0 | 60 |

TABLE 2
Treatments received for the cohorts

| | LHSC | LHSC | TCGA | TCGA | JHU | JHU | TMA | TMA | BD2Decide | BD2Decide | W&V | W&V |
|---|---|---|---|---|---|---|---|---|---|---|---|---|
| Treatment | (n) | (%) | (n) | (%) | (n) | (%) | (n) | (%) | (n) | (%) | (n) | (%) |
| Surgery + | 1 | 2.3 | 33 | 46.5 | 1 | 2.1 | 2 | 1 | 31 | 10.8 | 107 | 40.8 |
| Chemotherapy + | | | | | | | | | | | | |
| Radiation | | | | | | | | | | | | |
| Chemotherapy + | 30 | 69.7 | 0 | 0 | 11 | 23.4 | 103 | 52.2 | 182 | 63.7 | 53 | 20.2 |
| Radiation | | | | | | | | | | | | |
| Surgery + | 0 | 0 | 3 | 4.2 | 0 | 0 | 0 | 0 | 0 | 0 | 0 | 0 |
| Chemotherapy | | | | | | | | | | | | |
| Surgery + | 4 | 9.3 | 10 | 14.1 | 3 | 6.4 | 0 | 0 | 16 | 5.6 | 61 | 23.3 |
| Radiation | | | | | | | | | | | | |
| Chemotherapy | 0 | 0 | 0 | 0 | 1 | 2.1 | 0 | 0 | 0 | 0 | 0 | 0 |
| alone | | | | | | | | | | | | |
| Radiation | 4 | 9.3 | 0 | 0 | 4 | 8.5 | 88 | 44.7 | 47 | 16.4 | 0 | 0 |
| alone | | | | | | | | | | | | |
| Surgery | 4 | 9.3 | 10 | 14.1 | 27 | 57.4 | 2 | 1 | 9 | 3.2 | 29 | 11.1 |
| alone | | | | | | | | | | | | |
| Immunotherapy + | 0 | 0 | 1 | 1.4 | 0 | 0 | 0 | 0 | 0 | 0 | 0 | 0 |
| surgery | | | | | | | | | | | | |
| Unknown | 0 | 0 | 14 | 19.7 | 0 | 0 | 0 | 0 | 0 | 0 | 12 | 4.6 |
| No treatment | 0 | 0 | 0 | 0 | 0 | 0 | 2 | 1 | 1 | 0.3 | 0 | 0 |
| Total | 43 | 100 | 71 | 100 | 47 | 100 | 197 | 100 | 286 | 100 | 262 | 100 |

“W&V”: Washington University and Vanderbilt University

TABLE 3
Multivariate analysis of the UWO3 immune class.

| | Disease Free Survival | | |
|---|---|---|---|
| | HR (95% CI) | | P value |
| UWO3 Immune Class | | | |
| Mixed vs. Immune Rich | 6.4 | (2.21-18.7) | 0.0006 |
| Immune Desert vs. | 9.0 | (3.17-25.5) | 3.6 × 10⁻⁵ |
| Immune Rich | | | |
| Sex | | | |
| Female vs. Male | 1.29 | (0.63-2.63) | 0.47 |
| Age | 1.04 | (1.01-1.07) | 0.0035 |
| AJCC 8th edition stage | | | |
| Stage II vs. Stage I | 2.04 | (1.11-3.75) | 0.022 |
| Stage III vs. Stage I | 2.28 | (1.26-4.09) | 0.006 |
| Stage IV vs. Stage I | 5.41 × 10⁻⁷ | (0-Inf) | 1.00 |
| Smoking | | | |
| Yes vs. No | 1.43 | (0.79-2.57) | 0.24 |

AJCC: American joint committee on cancer. HR: hazard ratio. CI: confidence interval.

The above disclosure generally describes the present disclosure. Changes in form and substitution of equivalents are contemplated as circumstances may suggest or render expedient. Although specific terms have been employed herein, such terms are intended in a descriptive sense and not for purposes of limitation. Other variations and modifications of the disclosure are possible. As such modifications or variations are believed to be within the sphere and scope of the disclosure as defined by the claims appended hereto.

1: A method for determining a prognosis for a cancer patient, the prognosis predicts a probability of disease-free survival for the cancer patient, the method comprising:
(a) obtaining a sample having cancer cells from the cancer patient,
(b) measuring gene expression levels of ZAP70, IRF4 and CD3E in the sample,
(c) determining a tumor microenvironment (TME) subtype of the sample based on the gene expression levels of ZAP70, IRF4 and CD3E, the TME subtype being immune rich or non-immune rich, and
(d) determining, based on the determined TME subtype, that the cancer patient has an increased likelihood of overall survival if the sample has an immune rich TME subtype.

2: The method of claim 1, wherein the non-immune rich TME subtype includes an immune mixed TME subtype and an immune depleted TME subtype, and wherein the sample having the immune depleted TME subtype indicates that the cancer patient has a poor likelihood of overall survival, and the sample having the immune mixed TME subtype indicates that the cancer patient has worse likelihood of survival than then immune rich TME subtype and better likelihood of survival than the immune depleted TME subtype.

3: The method according to claim 1, wherein the method is done following a therapeutic treatment of the cancer in the cancer patient, and wherein the sample is obtained prior to or during the therapeutic treatment.

4: The method according to claim 1, wherein the method further comprises determining an expression score of the sample based on the gene expression levels measured in (b), and wherein the TME subtype is determined based on the expression score of the test cancer sample.

5: The method of claim 4, wherein the gene expression levels of step (b) is obtained by counting a number cells within the sample that test positive for the presence of ZAP70, IRF4 and CD3E, and wherein said number of positive cells is used to generate the expression score of the sample.

6: The method of claim 4, wherein the gene expression levels of step (b) is obtained by measuring the levels of RNA transcript of ZAP70, IRF4 and CD3E.

7: The method according to claim 4, wherein the expression score of the test cancer sample is determined using a known control reference value of each of the immune rich TME subtype and the non-immune rich TME subtype.

8: The method according to claim 4, wherein the expression score of the sample is determined using formula I: K1+abundance of ZAP70×K2+abundance of IRF4×K3+abundance of CD3E×K4 (I) wherein K1 is a constant between 2 and 2.5, K2 is a constant between −0.2 and −0.5, K3 is a constant between −0.1 and −0.15 and K4 is a constant between −0.2 and −0.3, and when the expression score of the sample is less or equal to 2 is indicative of the immune rich TME subtype, when the expression score of the sample is larger than 2 and less or equal to 2.5 is indicative of the immune mixed TME subtype as defined in claim 2, and when the expression score of the sample is larger than 2.5 is indicative of the immune depleted TME subtype as defined in claim 2.

9: The method of claim 8, wherein K1 is 2.23255813953488, K2 is −0.224179275535717, K3 is −0.137196384259042 and K4 is −0.273419927248369.

10: The method of claim 1, wherein step (b) further comprises measuring gene expression level of WDFY4 in the sample, and in step (c) determining the TME subtype of the sample is based on the gene expression levels of ZAP70, IRF4, CD3E and WDFY4.

11: The method of according to claim 2, wherein when the sample is determined to be immune depleted TME subtype, the method further comprises treating the cancer patient with at least one of chemotherapy, radiotherapy, and immunotherapy suitable for a poor prognosis.

12: The method of according to claim 1, wherein when the sample is determined to be immune rich TME subtype, the method further comprises treating the cancer patient with at least one of chemotherapy, radiotherapy and immunotherapy suitable for the favorable prognosis.

13: The method according to claim 1, wherein the cancer is human papillomavirus-driven (HPV⁺) head and neck squamous cell carcinoma (HNSCC), non-human papillomavirus driven (HPV−) HNSCC, endometrial cancer or cervical cancer.

14: The method according to claim 1, wherein the cancer is human papillomavirus-driven (HPV⁺) head and neck squamous cell carcinoma (HNSCC).

15: A method for determining a prognosis for a cancer patient, the method comprising:
(a) obtaining a sample comprising cancer cells from the cancer patient,
(b) measuring gene expression levels of ZAP70, IRF4 and CD3E in the sample, and
(c) determining a tumor microenvironment (TME) subtype of the sample by comparing the gene expression levels of (b) with known abundance reference gene expression values of ZAP70, IRF4 and CD3E in an immune rich TME subtype, and in a non-immune rich TME subtype, wherein the prognosis predicts a probability of disease-free survival for the cancer patient, and wherein determining that the test cancer tissue sample has the immune rich TME subtype indicates a favorable prognosis, and determining that the test cancer tissue sample has the non-immune rich TME subtype indicates a poor prognosis.

16: The method of claim 15, wherein the non-immune rich TME subtype includes an immune mixed TME subtype and an immune depleted TME subtype, and wherein the immune depleted TME subtype indicates a poor prognosis and the immune mixed TME subtype indicates worse prognosis than the immune rich TME subtype and better prognosis than the immune depleted TME subtype.

17: The method of claim 15, wherein step (b) further comprises measuring gene expression levels of WDFY4 in the sample, and step (c) comprises comparing the gene expression levels of (b) with known abundance reference gene expression values of ZAP70, IRF4, CD3E and WDFY4 in control samples of the immune rich TME subtype, the immune mixed TME subtype and the immune depleted TME subtype.

18: The method of claim 15, wherein the gene expression levels are obtained by counting the number of cells in the sample that stain positive for the presence of CD3, ZAP70 and IRF4.

19: The method of claim 17, wherein the gene expression levels are obtained by counting the number of cells in the sample that stain positive for the presence of CD3, ZAP70, IRF4 and WDFY4.

20: The method of claim 15, wherein the gene expression levels are obtained by measuring the levels of RNA transcript of ZAP70, IRF4 and CD3E.

21-37. (canceled)
